(19) United States
(12) Patent Application Publication
(10) Pub. No.: US 2011/0053964 A1
(43) Pub. Date: Mar. 3, 2011

(54) 4-AMINOQUINAZOLINE DERIVATIVES AND METHODS OF USE THEREOF

(76) Inventors: Roger Tung, Lexington, MA (US); Adam J. Morgan, Ashland, MA (US)

(21) Appl. No.: 12/879,905

(22) Filed: Sep. 10, 2010

Related U.S. Application Data
(63) Continuation-in-part of application No. 11/895,174, filed on Aug. 22, 2007.
(60) Provisional application No. 60/839,503, filed on Aug. 22, 2006.

(51) Int. Cl. A61K 31/517; C07D 405/10; A61P 35/00; A61P 35/02; C12N 5/02

(52) U.S. Cl. 514/266.24; 544/293; 435/375

(57) ABSTRACT

This invention relates to novel 4-aminoquinazolines and pharmaceutically acceptable salts thereof. This invention also provides compositions comprising a compound of this invention and the use of such compositions in methods of treating diseases and conditions that are beneficially treated by administering inhibitors of the EGFR and HER-2.

### RELATED APPLICATIONS

This application is a continuation-in-part of U.S. patent application Ser. No. 11/895,174, filed Aug. 22, 2007, which claims the benefit of U.S. Provisional Patent Application No. 60/839,503 filed Aug. 22, 2006, both of which are incorporated by reference herein in their entirety.

### FIELD OF THE INVENTION

This invention relates to novel 4-aminoquinazolines and pharmaceutically acceptable salts thereof. This invention also provides compositions comprising a compound of this invention and the use of such compositions in methods of treating diseases and conditions that are beneficially treated by administering inhibitors of the EGFR and HER2.

### BACKGROUND OF THE INVENTION

Lapatinib, also known as N-[3-Chloro-4-(3-fluorobenzyloxy)phenyl]-6-[5-[2-(methylsulfonyl)ethylaminomethyl]furan-2-yl]quinazolin-4-amine bis(4-methylbenzenesulfonate)hydrate, inhibits the tyrosine kinase activity of both the Epidermal Growth Factor Receptor (EGFR; ErbB1) and the human epidermal receptor Type 2 (HER2; ErbB2).

Lapatinib has been approved in the United States in combination with capecitabine (Xeloda®) for the treatment of patients with advanced or metastatic breast cancers whose tumor overexpress HER2 and who have failed prior therapy. Lapatinib has also been approved in the United States in combination with letrozole (Femara®) for the treatment of postmenopausal women with hormone receptor-positive metastatic breast cancer that overexpresses the HER2 receptor for whom hormonal therapy is indicated. In Europe, lapatinib has been approved in combination with an aromatase inhibitor for the treatment of postmenopausal women with hormone receptor-positive, HER2 over-expressing metastatic breast cancer and for whom chemotherapy is not intended.

Lapatinib is both metabolized by and also inhibits cytochrome P450 subtype 3A4 (CYP 3A4) at clinically relevant concentrations. The FDA approval label suggests avoiding co-dosing with strong CYP3A4 inhibitors or reducing the dose of lapatinib in patients requiring administration of compounds that are CYP3A4 inhibitors (http://www.fda.gov/cder/foi/label/2007/0220591b1.pdf). It is noteworthy that gastrointestinal toxicity, a clinically limiting aspect of the drug, appears to be related to the amount dosed rather than to plasma concentrations (Burris H A et al., J Clin Oncol 2005; 23:5305). This suggests that local drug concentrations in the gut are responsible for lapatinib's gastrointestinal toxicity and that increasing plasma concentrations for a given oral dose is likely to increase its therapeutic window and therefore enhance its utility without resulting in an associated increase in adverse side-effects.

Hepatotoxicity has been observed in clinical trials and post-marketing experience. The hepatotoxicity may be severe and deaths have been reported with lapatinib. See black box warning in the prescribing information (label) for TYKERB at http://us.gsk.com/products/assets/us_tykerb.pdf.

Compounds that are chemically related to lapatinib have also been described and have been shown to have potent tyrosine kinase inhibitory activity against ErbB1, ErbB2 and/or ErbB4 (HER4). See U.S. Pat. No. 6,727,256.

Despite the beneficial activities of lapatinib, there is a continuing need for new compounds to treat the aforementioned diseases and conditions, especially new compounds that are safer and well-tolerated.

### BRIEF DESCRIPTION OF THE FIGURES

FIG. 1 depicts the stability of various compounds of the invention in CYP3A4 SUPERSOMES™ as compared to lapatinib.

FIG. 2 depicts the pharmacokinetics of various compounds of the invention as compared to lapatinib after intravenous administration in rats.

FIG. 3 depicts a separate experiment examining the pharmacokinetics of various compounds of the invention as compared to lapatinib after intravenous administration in rats.

### DETAILED DESCRIPTION OF THE INVENTION

### Definitions

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein, e.g., a neoplasia).

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of lapatinib will inherently contain small amounts of deuterated and/or ¹³C-containing isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each position designated as having a deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation)

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. The term “isotopologue” refers to a species whose chemical structure differs from a specific compound of this invention only in the isotopic composition thereof.

The term “compound,” as used herein, is also intended to include any salts thereof.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, tosic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I or Ia), may contain an asymmetric carbon atom, for example, as the result of deuterium substitution or otherwise. As such, compounds of this invention can exist as either individual enantiomers, or mixtures of the two enantiomers,. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, for example less than 10% of other stereoisomers, for example less than 5% of other stereoisomers, for example less than 2% of other stereoisomers are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

Both “²H” and “D” refer to deuterium.

“Stereoisomer” refers to both enantiomers and diastereomers.

“Tert”, “^{t}”, and “t-” each refer to tertiary.

“US” refers to the United States of America.

“FDA” refers to Food and Drug Administration.

“NDA” refers to New Drug Application.

The term “isolated and purified” when used in conjunction with a compound refers to a compound that is isolated from another component or from other components of a naturally occurring source (such as a plant or animal cell) or from a synthetic organic chemical reaction mixture, and processed through one or more purifying steps that separate the compound of the invention from other molecules associated with it. When isolated and purified, a compound is at least about 95% pure. In one embodiment, the compound is at least about 98% pure. In another embodiment, the compound is at least about 99% pure.

“Neoplastic disease” means a disease that is caused by or results in inappropriately high levels of cell division, inappropriately low levels of apoptosis, or both. For example, cancer is an example of a proliferative disease. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

Throughout this specification, reference to “each Y” includes, independently, all “Y” groups (Y^{1a}, Y^{1b}, Y^{1c}, Y^{2a}, Y^{2b, Y}^{3a}, Y^{3b}, Y^{4a}, Y^{4b}, Y^{5a}, Y^{5b}) where applicable.

The term “heavy atom” refers to isotopes of higher atomic weight than the predominant naturally occurring isotope. The term “stable heavy atom” refers to non-radioactive heavy atoms.

### Therapeutic Compounds

The present invention provides novel 4-aminoquinazolines having advantageous biopharmaceutical properties useful for the treatment of neoplasia.

In one aspect, the invention provides a compound of formula I:

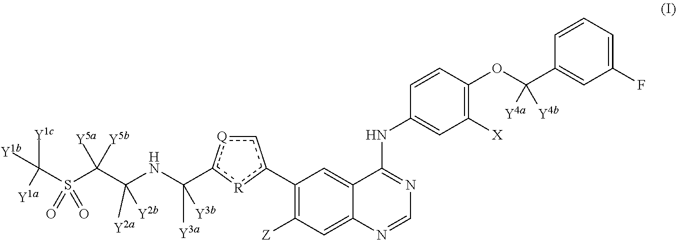

or a salt thereof; wherein:

R is oxygen, Q is carbon and the ring comprising R and Q is a furan; or

R is nitrogen, Q is sulfur and the ring comprising R and Q is a thiazole;

Z is hydrogen or fluorine;

X is chlorine or bromine;

each Y is independently selected from hydrogen and deuterium; and

at least one Y is deuterium.

In one selected embodiment, Z is fluorine.

In another embodiment, R is oxygen.

In yet another embodiment, X is chlorine.

In still another embodiment, Z is hydrogen.

In a selected embodiment, the invention provides a compound of Formula Ia:

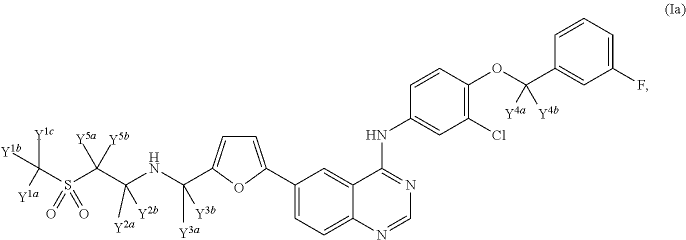

or a salt thereof; wherein each Y is defined as above for formula I.

In one embodiment of either Formula I or Ia, each Y bound to a common carbon atom is the same.

In another embodiment of Formula I or Ia, Y^{1a}, Y^{1b}, and Y^{1c} are simultaneously deuterium.

In yet another embodiment of Formula I or Ia, Y^{2a} and Y^{2b} are simultaneously deuterium. In a more specific embodiment, each Y bound to a common carbon atom is the same; Y^{2a} and Y^{2b} are simultaneously deuterium; and one or more of each Y¹, each Y³, each Y⁴ and each Y⁵ is deuterium.

In another embodiment of Formula I or Ia, Y^{3a} and Y^{3b} are simultaneously deuterium.

In still another embodiment of Formula I or Ia, Y^{4a} and Y^{4b} are simultaneously deuterium. In a more specific embodiment, each Y bound to a common carbon atom is the same; Y^{4a} and Y^{4b} are simultaneously deuterium; and one or more of each Y¹, each Y², each Y³ and each Y⁵ is deuterium. In another specific embodiment, each Y bound to a common carbon atom is the same; and Y^{2a}, Y^{2b}, Y^{4a} and Y^{4b} are simultaneously deuterium.

In yet another embodiment of Formula I or Ia, Y^{5a} and Y^{5b} are simultaneously deuterium.

In still another embodiment of Formula I or Ia, the compound contains at least two, three, four, five, six, seven, eight, or nine deuterium.

In one embodiment, the compound of Formula I or Ia is isolated. In a more particular embodiment, the compound of Formula I or Ia is isolated and purified.

In another embodiment, the salt of a compound of Formula I or Ia is a pharmaceutically acceptable salt. In a more specific embodiment, the pharmaceutically acceptable salt of a compound of Formula I or Ia is a tosylate salt.

In yet another embodiment, the compound is a compound of Formula Ia selected from any one of the compounds (Cmpd) set forth in Table 1 (below):

TABLE 1
Exemplary Embodiments of Formula Ia

| Cmpd | Y^{1a} | Y^{1b} | Y^{1c} | Y^{2a} | Y^{2b} | Y^{3a} | Y^{3b} | Y^{4a} | Y^{4b} | Y^{5a} | Y^{5b} |
|---|---|---|---|---|---|---|---|---|---|---|---|
| 100 | H | H | H | D | D | H | H | H | H | H | H |
| 101 | H | H | H | D | D | D | D | H | H | H | H |
| 102 | D | D | D | D | D | D | D | H | H | D | D |
| 103 | D | D | D | D | D | H | H | H | H | D | D |
| 104 | H | H | H | D | D | D | D | D | D | H | H |
| 105 | D | D | D | D | D | D | D | D | D | D | D |
| 106 | H | H | H | H | H | D | D | D | D | H | H |
| 107 | H | H | H | H | H | H | H | D | D | H | H |
| 108 | H | H | H | D | D | H | H | D | D | H | H |

In one aspect, the invention provides a compound of Formula II:

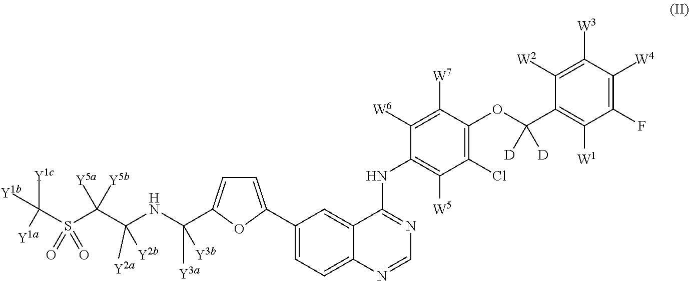

or salt thereof, wherein:

each W and each Y is independently selected from hydrogen and deuterium; and

at least one W is deuterium and at least one of Y and W is hydrogen.

In one embodiment of Formula II, each of W¹ and W² is deuterium.

In one embodiment of Formula II, each of W¹, W², W⁵, W⁶, and W⁷ is deuterium.

In one embodiment of Formula II, each of W¹, W², W³ and W⁴ is deuterium. In one aspect of this embodiment each of W⁵, W⁶ and W⁷ is hydrogen. In another aspect of this embodiment each of W⁵, W⁶ and W⁷ is deuterium.

In one embodiment of Formula II, each of W⁵, W⁶ and W⁷ is deuterium. In one aspect of this embodiment, each of W¹, W², W³ and W⁴ is hydrogen.

In one embodiment of Formula II, each Y bound to a common carbon atom is the same.

In another embodiment of Formula II, Y^{1a}, Y^{1b}, and Y^{1c} are simultaneously deuterium.

In yet another embodiment of Formula II, Y^{2a} and Y^{2b} are simultaneously deuterium. In a more specific embodiment, each Y bound to a common carbon atom is the same; Y^{2a} and Y^{2b} are simultaneously deuterium; and one or more of each Y¹, each Y³ and each Y⁵ is deuterium.

In another embodiment of Formula II, Y^{3a} and Y^{3b} are simultaneously deuterium.

In yet another embodiment of Formula II, Y^{5a} and Y^{5b} are simultaneously deuterium.

In another embodiment, the salt of a compound of Formula II is a pharmaceutically acceptable salt. In a more specific embodiment, the pharmaceutically acceptable salt of a compound of Formula II is a tosylate salt.

Examples of specific compounds of Formula II are shown in Table 2 (below):

TABLE 2
Exemplary Embodiments of Formula II

| | | | | | | | | each of | | | |
|---|---|---|---|---|---|---|---|---|---|---|---|
| | | | | | | | | W¹, W², | each of | | |
| | | | | | | | | W³ and | W⁵, W⁶, | | |
| Cmpd | Y^{1a} | Y^{1b} | Y^{1c} | Y^{2a} | Y^{2b} | Y^{3a} | Y^{3b} | W⁴ | and W⁷ | Y^{5a} | Y^{5b} |
| 200 | H | H | H | H | H | H | H | D | H | H | H |
| 201 | H | H | H | D | D | D | D | D | H | H | H |
| 202 | D | D | D | D | D | D | D | D | H | D | D |
| 203 | D | D | D | D | D | H | H | D | H | D | D |
| 204 | H | H | H | H | H | D | D | D | H | H | H |
| 205 | H | H | H | D | D | H | H | D | H | H | H |
| 300 | H | H | H | H | H | H | H | H | D | H | H |
| 301 | H | H | H | D | D | D | D | H | D | H | H |
| 302 | D | D | D | D | D | D | D | H | D | D | D |
| 303 | D | D | D | D | D | H | H | H | D | D | D |
| 304 | H | H | H | H | H | D | D | H | D | H | H |
| 305 | H | H | H | D | D | H | H | H | D | H | H |
| 400 | H | H | H | H | H | H | H | D | D | H | H |
| 401 | H | H | H | D | D | D | D | D | D | H | H |
| 402 | D | D | D | D | D | H | H | D | D | D | D |
| 403 | H | H | H | H | H | D | D | D | D | H | H |
| 404 | H | H | H | D | D | H | H | D | D | H | H |

or a salt thereof.

In one embodiment, the compound of formula II is a compound of Formula IIb:

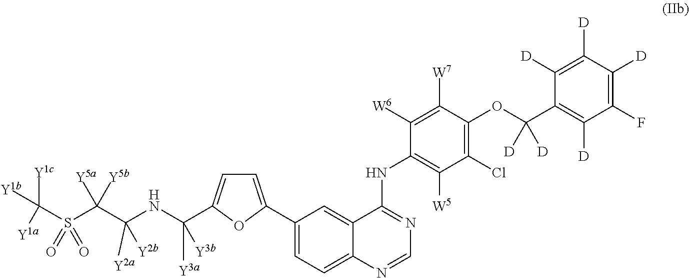

or a salt thereof, wherein

each W and each Y is independently selected from hydrogen and deuterium.

In one embodiment of formula IIb, the compound is

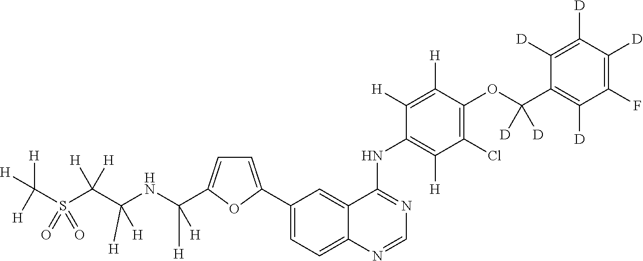

or a salt thereof, wherein any atom not designated as deuterium is present at its natural isotopic abundance.

In one embodiment, the compound of formula II is a compound of Formula IIc:

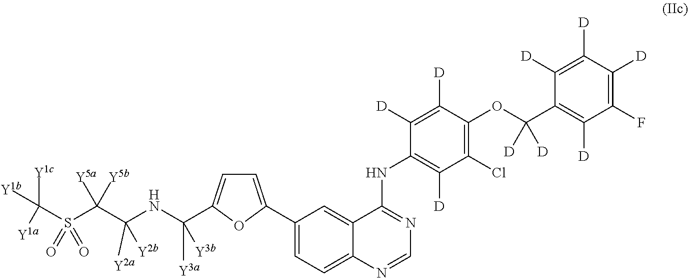

or a salt thereof wherein each Y is independently hydrogen or deuterium.

In one aspect, the invention provides a compound of Formula IIa:

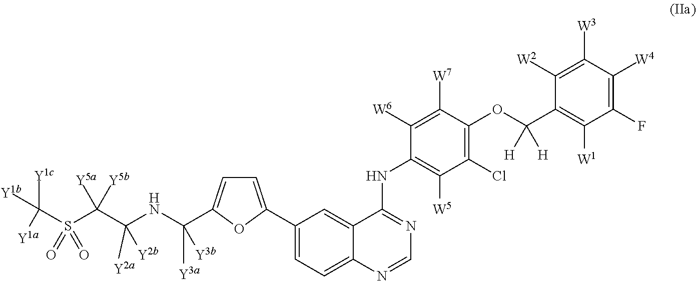

or salt thereof, wherein:

each Y is independently selected from hydrogen and deuterium; and

each W is independently selected from hydrogen and deuterium; and

at least one W is deuterium;

provided that if either (i) W¹, W², W³ and W⁴ are each deuterium or (ii) W⁵, W⁶, and W⁷ are each deuterium, then at least one remaining W or at least one Y is deuterium.

In one embodiment of Formula IIa, each of W¹, W² is deuterium.

In one embodiment of Formula IIa, each of W¹, W², W³, and W⁴ is deuterium.

In one embodiment of Formula IIa, each of W¹, W², W⁵, W⁶, and W⁷ is deuterium.

In a further embodiment of Formula IIa, each of W¹, W², W³, W⁴, W⁵, W⁶, and W⁷ is deuterium.

In one embodiment of Formula IIa, each of W⁵, W⁶, and W⁷ is deuterium.

In one embodiment of Formula IIa, IIb or IIc, each Y bound to a common carbon atom is the same.

In another embodiment of Formula IIa, IIb or IIc, Y^{1a}, Y^{1b}, and Y^{1c} are simultaneously deuterium.

In yet another embodiment of Formula IIa, IIb or IIc, Y^{2a} and Y^{2b} are simultaneously deuterium. In a more specific embodiment, each Y bound to a common carbon atom is the same; Y^{2a} and Y^{2b} are simultaneously deuterium; and one or more of each Y¹, each Y³ and each Y⁵ is deuterium.

In another embodiment of Formula IIa, IIb or IIc, Y^{3a} and Y^{3b} are simultaneously deuterium.

In yet another embodiment of Formula IIa, IIb or IIc, Y^{5a} and Y^{5b} are simultaneously deuterium.

In another embodiment, the salt of a compound of Formula IIa, IIb or IIc, is a pharmaceutically acceptable salt. In a more specific embodiment, the pharmaceutically acceptable salt of a compound of Formula IIa, IIb or IIc, is a tosylate salt. Examples of specific compounds of Formula IIa are shown in Table 3 (below):

TABLE 3
Exemplary Embodiments of Formula IIa

| | | | | | | | | each of | | | |
|---|---|---|---|---|---|---|---|---|---|---|---|
| | | | | | | | | W¹, W², | each of | | |
| | | | | | | | | W³ and | W⁵, W⁶, | | |
| Cmpd | Y^{1a} | Y^{1b} | Y^{1c} | Y^{2a} | Y^{2b} | Y^{3a} | Y^{3b} | W⁴ | and W⁷ | Y^{5a} | Y^{5b} |
| 501 | H | H | H | D | D | D | D | D | H | H | H |
| 502 | D | D | D | D | D | D | D | D | H | D | D |
| 503 | D | D | D | D | D | H | H | D | H | D | D |
| 504 | H | H | H | H | H | D | D | D | H | H | H |
| 505 | H | H | H | D | D | H | H | D | H | H | H |
| 601 | H | H | H | D | D | D | D | H | D | H | H |
| 602 | D | D | D | D | D | D | D | H | D | D | D |
| 603 | D | D | D | D | D | H | H | H | D | D | D |
| 604 | H | H | H | H | H | D | D | H | D | H | H |
| 605 | H | H | H | D | D | H | H | H | D | H | H |
| 700 | H | H | H | H | H | H | H | D | D | H | H |
| 701 | H | H | H | D | D | D | D | D | D | H | H |
| 702 | D | D | D | D | D | H | H | D | D | D | D |
| 703 | H | H | H | H | H | D | D | D | D | H | H |
| 704 | H | H | H | D | D | H | H | D | D | H | H |
| 705 | D | D | D | D | D | D | D | D | D | D | D |

or a salt thereof.

In one embodiment, any atom not designated as deuterium in any of the embodiments set forth above is present at its natural isotopic abundance.

Combinations of substituents and variables envisioned by this invention are only those that result in the formation of stable compounds.

In another aspect, the invention provides a mixture containing or consisting essentially of a compound of formula I; and a lighter isotopologue of the compound of formula I, where at least 50%, 60%, 75%, 80%, 85%, 90%, or 95% of the mixture is the compound of formula I.

### Compound Synthesis

Compounds of formula I, Ia, II, IIa, IIb or IIc or salts thereof can be made by means known in the art of organic synthesis. For instance, routes to the all-hydrogen isotopologues of compounds of this invention and intermediates thereof are described in U.S. Pat. No. 6,727,256. Methods of incorporating deuterium in target compounds are extensively documented. See, for instance, The Journal of Labelled Compounds and Radiopharmaceuticals (John Wiley & Sons), most issues of which contain detailed experimental descriptions on specific incorporation of deuterium into bioactive small organic molecules. See also, for instance, Leis H J, Curr Org Chem, 1998, 2:131 and reference therein, and Moebius G, Zfi-Mitteilungen 1989, 150:297. Suitable commercial supplies of deuterium-labeled reagents include, among others, Isotec, Inc. (Miamisburg, Ohio); Cambridge Isotope Laboratories (Andover, Mass.); ICON Services Inc. (Summit, N.J.); and C/D/N Isotopes, Inc. (Pointe-Claire, Quebec, Canada). Certain intermediates can be used with or without purification (e.g., filtration, distillation, sublimation, crystallization, trituration, solid phase extraction, and chromatography). Illustrative methods of synthesis are shown and described below and in the Examples herein. The invention further provides intermediates useful for synthesizing a compound of Formula I, Ia, II, IIa, IIb or IIc.

A convenient method for synthesizing compounds of Formula I, Ia or II or IIa is depicted in Scheme A, wherein Q, R, X, Z and each Y is as defined above:

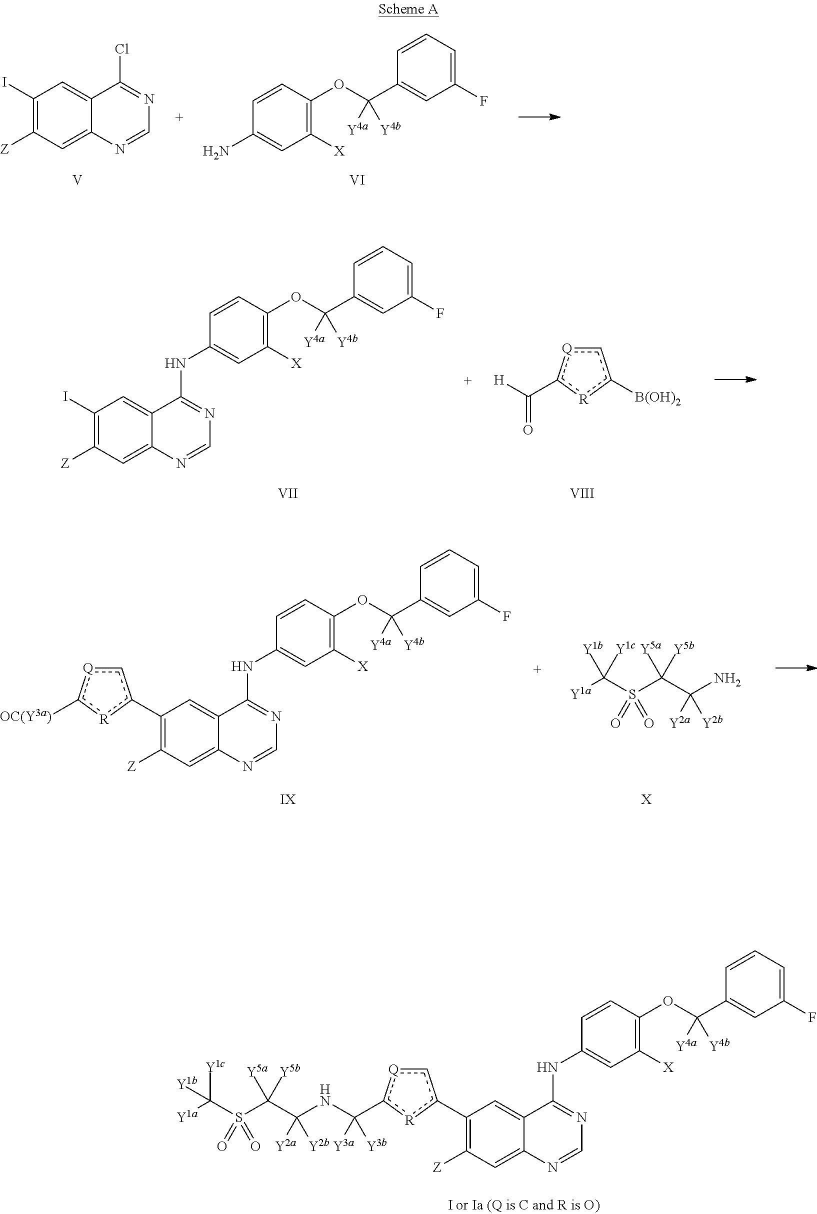

As shown in Scheme A, reaction of quinazoline V with substituted 4-(benzyloxy)-aniline VI produces compound VII, which can then be coupled (e.g., in the presence of a palladium catalyst) with boronic acid (VIII) to yield intermediate IX. Reductive amination of IX with amine X yields a compound of Formula I or Ia. In one embodiment, Y^{3a} is H.

A convenient method for synthesizing compounds of Formula II or IIa or IIb or IIc is depicted in Scheme B.

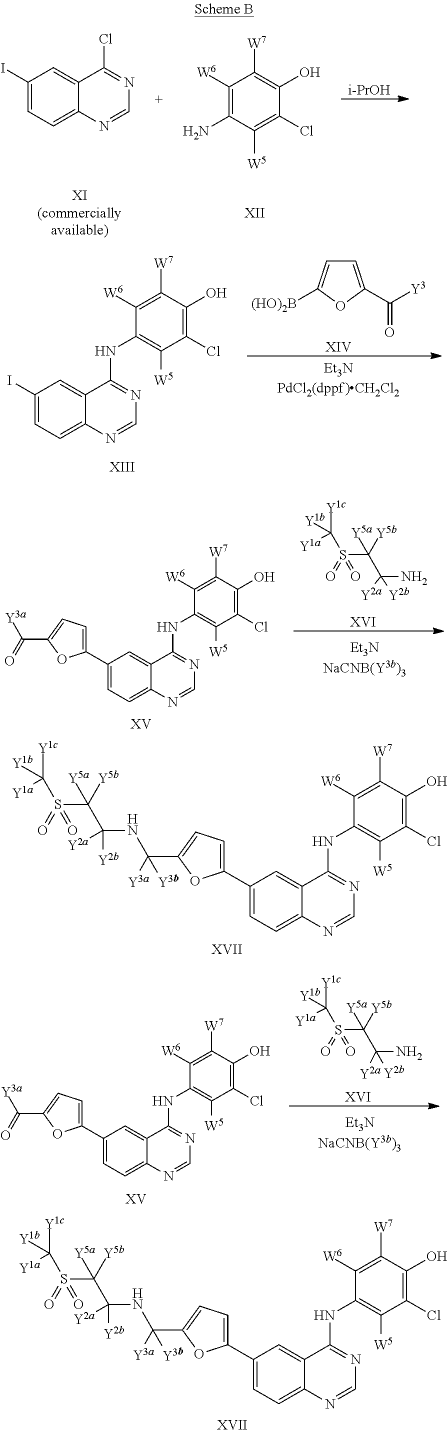

As shown in Scheme B, reaction of quinazoline XI with appropriately deuterated 4-amino-2-chlorophenol XII provides XIII, which is then coupled (for example, in the presence of a palladium catalyst) with appropriately deuterated XIV to yield XV. Reductive amination of XV with appropriately deuterated amine XVI yields XVII. After a protection step to give XVIII, treatment with appropriately deuterated 3-fluorobenzyl bromide XIX affords a compound of Formula II or IIa.

Convenient preparations of examples of compounds XII and XIX are shown in Scheme C and D, respectively.

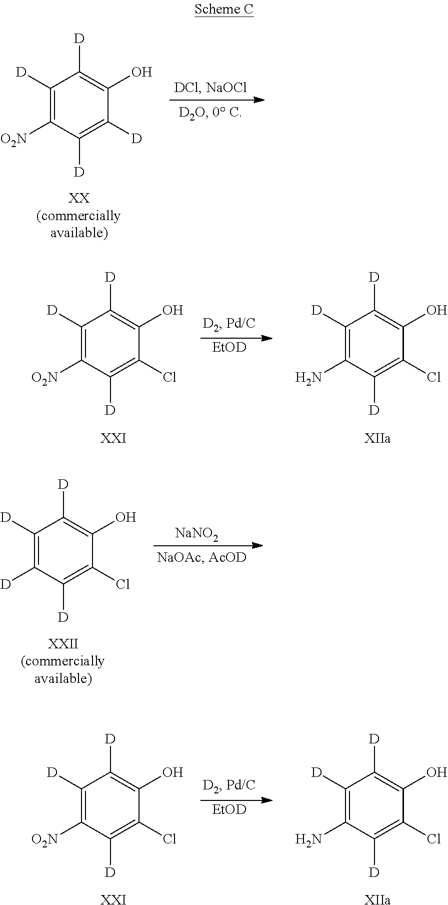

In Scheme C, compound XIIa is prepared from either (a) XX, in a manner analogous to the one described in Russian patent publication RU 2266877, or (b) XXII, in a manner analogous to Jin et al., Biorg. Med. Chem. Lett. 2006, 16, 5864-69. In (a), XX is treated with sodium hypochlorite to provide XXI, followed by reduction of XXI with D₂ and Pd/C to afford XIIa. In (b), XXII is treated with sodium nitrite to give XXI, followed by reduction and aqueous work-up to XIIa in the same way as described in (a).

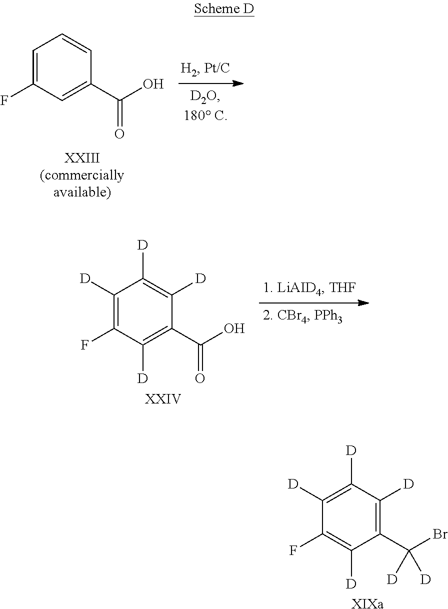

In Scheme D, compound XIXa is prepared from 3-fluorobenzoic acid XXIII in a manner analogous to the one described in Sajiki et al., Tetrahedron Lett. 2005, 45, 6995-98. XXIII is treated with H₂ and Pd/C followed by D₂O to provide XXIV, which is then treated with LiAlD₄ followed by CBr₄/PPh₃ to afford XIXa.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Additional methods of synthesizing compounds of this invention and their synthetic precursors, including those within routes not explicitly shown in schemes herein, are within the means of chemists of ordinary skill in the art. Methods for optimizing reaction conditions and, if necessary, minimizing competing by-products, are known in the art. In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database.

The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

### Pharmaceutical Compositions

The invention also provides compositions comprising an effective amount of a compound of Formula I or Ia or II or IIa or IIb or IIc, or a pharmaceutically acceptable salt thereof; and an acceptable carrier. In one embodiment, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament. In one embodiment, the compositions are pyrogen-free.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including for instance subcutaneous, intramuscular, intravenous, intrathecal and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy (20th ed.), ed. A. R. Gennaro, Lippincott Williams & Wilkins, 2000; and Encyclopedia of Pharmaceutical Technology, eds. J. Swarbrick and J. C. Boylan, 1988-1999, Marcel Dekker, New York.

For therapeutic uses, the compositions comprising compounds of formula I, Ia or II or IIa or IIb or IIc are obtained using the methods disclosed herein. Pharmaceutical compositions comprising such compounds may be administered systemically, for example, formulated in a pharmaceutically-acceptable buffer, such as physiological saline. Routes of administration include, for example, subcutaneous, intravenous, intraperitoneally, intramuscular, or intradermal injections that provide continuous, sustained levels of the drug in the patient. Treatment of human patients or other animals can be carried out using a therapeutically effective amount of a compound of formula I, Ia or II or IIa or IIb or IIc in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of a neoplastic disease. Generally, amounts will be in the range of those used for other agents used in the treatment of other neoplastic diseases, such as breast cancer, including metastatic cancer, although in certain instances lower amounts will be needed because of the decreased oxidation and increased half-life of the compound. A compound is administered at a dosage that controls the clinical or physiological symptoms of a neoplastic disease as determined by a diagnostic method known to one skilled in the art.

### Formulation of Pharmaceutical Compositions

The administration of a compound of formula I, Ia or II or IIa or IIb or IIc for the treatment of a neoplastic disease may be by any suitable means that results in a concentration of the therapeutic that, optionally combined with other components, is effective in ameliorating, reducing, or stabilizing a neoplastic disease, such as breast cancer, particularly metastatic breast cancer. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the tissue to be treated; (v) formulations that allow for convenient dosing, such that doses are administered, for example, once every day; once every 2 or 3 days, or once per week or per two weeks; and (vi) formulations that target a neoplastic disease by using carriers or chemical derivatives to deliver the therapeutic agent to a particular cell type, such as a neoplastic cell present in breast tissue or a cell that has metastasized from a primary cancer site. For some applications, controlled release formulations can contribute to the reduced rate of metabolism of the therapeutic compound and obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation or it may be presented as a dry powder to be reconstituted with water or another suitable vehicle before use. Apart from the active compounds of formula I, Ia or II or IIa or IIb or IIc therapeutic(s), the composition may include suitable parenterally acceptable carriers and/or excipients. The active chemotherapeutic(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

The compound of this invention may be mixed together in the tablet with one or more active therapeutics for the treatment of neoplasia, or the two or more active therapeutic may be partitioned within the tablet. In one example, the first active chemotherapeutic is contained on the inside of the tablet, and the second active therapeutic is on the outside, such that a substantial portion of the second active therapeutic is released prior to the release of the first therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release an active therapeutic described herein by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds of this invention may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosage

Pharmaceutical compositions may be presented in unit dose forms containing a predetermined amount of active ingredient per unit dose. Such a unit may contain, for example, 0.5 mg to 1200 mg, in another embodiment 1 mg to 1000 mg, in another embodiment 5 mg to 400 mg of a compound of formula I or Ia, depending on the condition being treated, the route of administration and the age, weight and condition of the patient, or pharmaceutical formulations may be presented in unit dose forms containing a predetermined amount of active ingredient per unit dose. For example, a unit dosage formulation of a compound of this invention may contain from about 100 mg to about 400 mg; or from about 150 mg to about 350 mg; or from about 200 mg to about 300 mg; or from about 225 mg to about 275 mg of a compound of formula I or Ia or II or IIa or IIb or IIc; or about 250 mg of a compound of formula I or Ia or II or IIa or IIb or IIc. One or more unit doses may be administered in a day, such as one, two, three, four, five, or six unit doses. For example, a five unit doses containing from about 225 mg to about 275 mg may be administered once a day. This corresponds to a daily dose of from about 1125 to about 1375 mg a day, such as about 1250 mg a day. As another example, a unit dose containing from about 225 mg to about 275 mg may be administered six times a day. This corresponds to a daily dose of from about 1350 to about1650 mg a day, such as about 1500 mg a day, Some unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

In any of the formulations set forth above, the compound of formula I or Ia or II or IIa or IIb or IIc or a pharmaceutically acceptable salt thereof may be combined with one or more second therapeutic agents in a single dosage form. Such second therapeutic agents include, but are not limited to, other anti-neoplastic agents and immunosuppressant. Examples of second therapeutic agents useful in such combination dosage forms include, but are not limited to, capecitabine, pazopanib, trastuzumab, docetaxel, letrozole, tamoxifen, fulvestrant, paclitaxel, carboplatin, bevacizumab, doxorubicin, cyclophosphamide, cisplatin, vinorelbine, everolimus, valproic acid, topotecan, oxaliplatin and gemcitabine. As a more specific example, the second therapeutic agent is capecitabine or letrozole.

### Therapeutic Methods

In one embodiment, the invention provides a method of inhibiting ErbB-1, ErbB-2, or ErbB-4-associated protein kinase activity in a cell comprising the step of contacting the cell with a compound of Formula I or Ia or II or IIa or IIb or IIc or a pharmaceutically acceptable salt thereof.

In another embodiment, the present invention provides methods of treating a subject suffering from or susceptible to a neoplastic disease. In one embodiment, the subject is a patient in need of such treatment. While compounds of Formula I or Ia or II or IIa or IIb or IIc are particularly useful for the treatment of breast cancer, particularly metastic breast cancer, the invention is not so limited. Illustrative neoplasms for which the invention can be used include, but are not limited to, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

In a specific embodiment, the subject is suffering from or susceptible to a breast cancer, esophageal adenocarcinoma, esophageal squamous cell carcinoma, cervical cancer, head and neck cancer, solid tumors, non-Hodgkins' Lymphoma, gastric cancer, ovarian cancer, peritoneal cancer, Brain and CNS tumors (glioma, glioblastoma multiforme, gliosarcoma), prostate cancer, endometrial cancer, colorectal cancer, non-small cell lung cancer, liver cancer, renal cancer, pancreatic cancer.

In another aspect of the present invention, there is provided a method of treating an erbB2, erbB4, or EGF (erbB1) receptor positive neoplasia in a mammal. In a specific embodiment, the subject is suffering from or susceptible to an erbB positive breast cancer. In a more specific embodiment, the breast cancer is erbB2, erbB4, or EGF receptor positive or overexpressing. In an even more specific embodiment, the breast cancer is erbB2, or EGF receptor positive. In another more specific embodiment, the breast cancer is not responsive to convention chemotherapies, and/or disorders or symptoms thereof. These methods can comprise administering a therapeutically effective amount of a pharmaceutical composition comprising a compound of Formula I or Ia or II or IIa or IIb or IIc or a pharmaceutically acceptable salt thereof to a subject (e.g., a mammal such as a human) in need thereof. A “therapeutically effective amount” of a compound herein is an amount sufficient to treat the disease or disorder or symptom thereof.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method). Identifying a subject “at risk” or susceptible for a disease, disorder, or symptom can be achieved by any objective or subjective determination by a diagnostic test or opinion of a subject or health care provider (e.g., genetic test, enzyme or protein marker (such as a phosphorylated EGF receptor, c-ErbB-2, or c-erbB-4), family history, and the like).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

### Combination Therapies

Optionally, a compound of the present invention is administered in combination with any other standard active anti-neoplastic therapy. Such therapies are known to the skilled artisan and include anti-neoplastic therapy, combination therapy with other chemotherapeutic, hormonal, antibody or immunosuppressive agents, as well as surgical and/or radiation treatments.

Anti-neoplastic therapies are described for instance in International Application No. PCT US 02/01130, filed Jan. 14, 2002, which describes anti-neoplastic therapies including, but not limited to, anti-microtubule agents, such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclines, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids. Diterpenoids, which are derived from natural sources, are phase specific anti-cancer agents that operate at the G2/M phases of the cell cycle. It is believed that the diterpenoids stabilize the [beta]-tubulin subunit of the microtubules, by binding with this protein. Disassembly of the protein appears is inhibited with mitosis being arrested and cell death following. Examples of diterpenoids include, but are not limited to, paclitaxel and its analog docetaxel. Paclitaxel, 5[beta],20-epoxy-1,2[alpha],4,7,[beta]10[beta],13[alpha]-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. Docetaxel, (2R,35)-N-carboxy-3-phenylisoserine,N-tert-butyl ester, 13-ester with 5[beta]-20-epoxy-1,2[alpha],4,7[beta],10[beta],13[alpha]-hexahydroxytax-11-en-9-one 4-acetate 2-benzoate, trihydrate; is commercially available as an injectable solution as TAXOTERE®. Vinca alkaloids are phase specific anti-neoplastic agents derived from the periwinkle plant. Examples of vinca alkaloids include, but are not limited to, vinblastine, vincristine, and vinorelbine.

Vinblastine, vincaleukoblastine sulfate, is commercially available as VELBAN® as an injectable solution. Vincristine, vincaleukoblastine, 22-oxo-, sulfate, is commercially available as ONCOVIN® as an injectable solution. Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Platinum coordination complexes are non-phase specific anti-cancer agents, which are interactive with DNA. Examples of platinum coordination complexes include, but are not limited to, cisplatin, oxaliplatin and carboplatin. Cisplatin, cis-diamminedichloroplatinum, is commercially available as PLATINOL® as an injectable solution. Carboplatin, platinum, diammine [1,1-cyclobutane-dicarboxylate(2-)-O,O′], is commercially available as PARAPLATIN® as an injectable solution. Alkylating agents are non-phase anti-cancer specific agents and strong electrophiles. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]benzenebutanoic acid, is commercially available as LEUKERAN® tablets. Busulfan, 1,4-butanediol dimethanesulfonate, is commercially available as MYLERAN® tablets. Carmustine, 1,3-[bis(2-chloroethyl)-1-nitrosourea, is commercially available as single vials of lyophilized material as BiCNU®. Dacarbazine, 5-(3,3-dimethyl-1-triazeno)-imidazole-4-carboxamide, is commercially available as single vials of material as DTIC-Dome®.

Antibiotic anti-neoplastics are non-phase specific agents, which bind or intercalate with DNA. Examples of antibiotic anti-neoplastic agents include, but are not limited to, actinomycins such as dactinomycin, anthracyclines such as daunorubicin and doxorubicin; and bleomycins. Dactinomycin, also know as Actinomycin D, is commercially available in injectable form as COSMEGEN®. Daunorubicin, (8S-cis-)-8-acetyl-10-[(3-amino-2,3,6-trideoxy-[alpha]-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Doxorubicin, (8S,10S)-10-[(3-amino-2,3,6-trideoxy-[alpha]-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Bleomycin, a mixture of cytotoxic glycopeptide antibiotics isolated from a strain of Streptomyces verticillus, is commercially available as BLENOXANE®.

Topoisomerase II inhibitors include, but are not limited to, epipodophyllotoxins. Examples of epipodophyllotoxins include, but are not limited to, etoposide and teniposide. Etoposide, 4′-demethyl-epipodophyllotoxin 9[4,6-0-(R)-ethylidene-[beta]-D-glucopyranoside], is commercially available as an injectable solution or capsules as VePESID® and is commonly known as VP-16. Teniposide, 4′-demethyl-epipodophyllotoxin 9[4,6-0-(R)-thenylidene-[beta]-D-glucopyranoside], is commercially available as an injectable solution as VUMON® and is commonly known as VM-26.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Examples of antimetabolite anti-neoplastic agents include, but are not limited to, fluorouracil, methotrexate, cytarabine, mercaptopurine, thioguanine, and gemcitabine. 5-fluorouracil, 5-fluoro-2,4-(1H,3H) pyrimidinedione, is commercially available as fluorouracil. Other fluoropyrimidine analogs include 5-fluoro deoxyuridine (floxuridine) and 5-fluorodeoxyuridine monophosphate. P Cytarabine, 4-amino-1-[beta]-D-arabinofuranosyl-2 (1H)-pyrimidinone, is commercially available as CYTOSAR-U® and is commonly known as Ara-C. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. A useful mercaptopurine analog is azathioprine. Thioguanine, 2-amino-1,7-dihydro-6H-purine-6-thione, is commercially available as TABLOID®. Other purine analogs include pentostatin, erythrohydroxynonyladenine, fludarabine phosphate, and cladribine. Gemcitabine, 2′-deoxy-2′,2′-difluorocytidine monohydrochloride ([beta]-isomer), is commercially available as GEMZAR®.

Methotrexate, N-[4[[(2,4-diamino-6-pteridinyl) methyl]methylamino]benzoyl]-L-glutamic acid, is commercially available as methotrexate sodium.

Camptothecins, including, camptothecin and camptothecin derivatives are available or under development as Topoisomerase I inhibitors. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino)carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Also useful, is the camptothecin derivative currently under development, including the racemic mixture (R,S) form as well as the R and S enantiomers: EMI5.0 known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrozole, vorozole, and exemestane useful in the treatment of adrenocortical carcinoma and hormone dependent breast carcinoma containing estrogen receptors; progestins such as megestrol acetate useful in the treatment of hormone dependent breast cancer and endometrial carcinoma; estrogens, androgens, and anti-androgens such as flutamide, nilutamide, bicalutamide, cyproterone acetate and 5[alpha]-reductases such as finasteride and dutasteride, useful in the treatment of prostatic carcinoma and benign prostatic hypertrophy; anti-estrogens such as tamoxifen, toremifene, raloxifene, droloxifene, iodoxifene, and fulvestrant, as well as selective estrogen receptor modulators (SERMS), such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutenizing hormone (LH) and/or follicle stimulating hormone (FSH) for the treatment prostatic carcinoma, for instance, LHRH agonists and antagonists such as goserelin acetate and leuprolide.

Monoclonal antibodies useful in treating neoplasias include trastuzumab (HERCEPTIN®) and anti-Her2 antibody and bevacizumab (AVASTIN®) and anti-VEGF antibody. Other anti-neoplastic agents useful in combination with the compounds of this invention include pazopanib, a VEGF inhibitor, and valproic acid which is believe to anti-angiogenesis properties.

Combination therapies according to the present invention thus include the administration of at least one compound of formula (I) or Ia or II or IIa or IIb or IIc or a pharmaceutically acceptable salt thereof as well as optional use of other therapeutic agents including other anti-neoplastic agents, such as the immunosuppressant everolimus. Such combination of agents may be administered together or separately and, when administered separately this may occur simultaneously or sequentially in any order, both close and remote in time. The amounts of the compound of formula (I) or Ia or II or IIa or IIb or IIc or a pharmaceutically acceptable salt thereof and the other pharmaceutically active agent(s) the relative timings of administration will be selected in order to achieve the desired combined therapeutic effect.

In one embodiment, the method of treating a subject suffering from or susceptible to a cancer comprises the additional step of administering to the subject in need thereof a second therapy selected from an anti-neoplastic therapy other than a compound of Formula I or Ia or II or IIa or IIb or IIc or a pharmaceutically acceptable salt thereof, and an immunosuppressant.

In one specific embodiment, the subject is suffering from or susceptible to breast cancer and the second therapy is selected from capecitabine, pazopanib, trastuzumab, docetaxel, letrozole, tamoxifen, fulvestrant, paclitaxel, carboplatin, bevacizumab, doxorubicin and cyclophosphamide. In a more specific aspect, the second therapeutic agent is capecitabine, which may be administered together with or separately from the compound of formula I, Ia or II or IIa or IIb or IIc or a pharmaceutically acceptable salt thereof. When capecitabine and the compound of formula I, Ia or II or IIa or IIb or IIc or a pharmaceutically acceptable salt thereof are administered separately, this may occur simultaneously or sequentially in any order. In an example of this aspect, the compound of formula I, Ia or II or IIa or IIb or IIc or a pharmaceutically acceptable salt thereof is administered without food and capecitabine is administered with food.

In another specific embodiment, the subject is suffering from or susceptible to cervical cancer and the second therapy is pazopanib.

In still another specific embodiment, the subject is suffering from or susceptible to head and neck cancer and the second therapy is selected from radiation treatment and cisplatin.

In another specific embodiment, the subject is suffering from or susceptible to solid tumors and the second therapy is selected from vinorelbine, everolimus, paclitaxel, valproic acid, docetaxel and topotecan.

In another specific embodiment, the subject is suffering from or susceptible to non-Hodgkin's lymphoma and the second therapy is everolimus.

In another specific embodiment, the subject is suffering from or susceptible to gastric cancer and the second therapy is paclitaxel.

In another specific embodiment, the subject is suffering from or susceptible to ovarian cancer and the second therapy is selected from carboplatin and topotecan.

In another specific embodiment, the subject is suffering from or susceptible to malignant glioma and the second therapy is pazopanib.

In another specific embodiment, the subject is suffering from or susceptible to peritoneal cancer and the second therapy is topotecan.

In another specific embodiment, the subject is suffering from or susceptible to pancreatic cancer and the second therapy is selected from oxaliplatin and gemcitabine.

### Examples

### Example 1

### Preparation of Non-Deuterated Intermediate 17

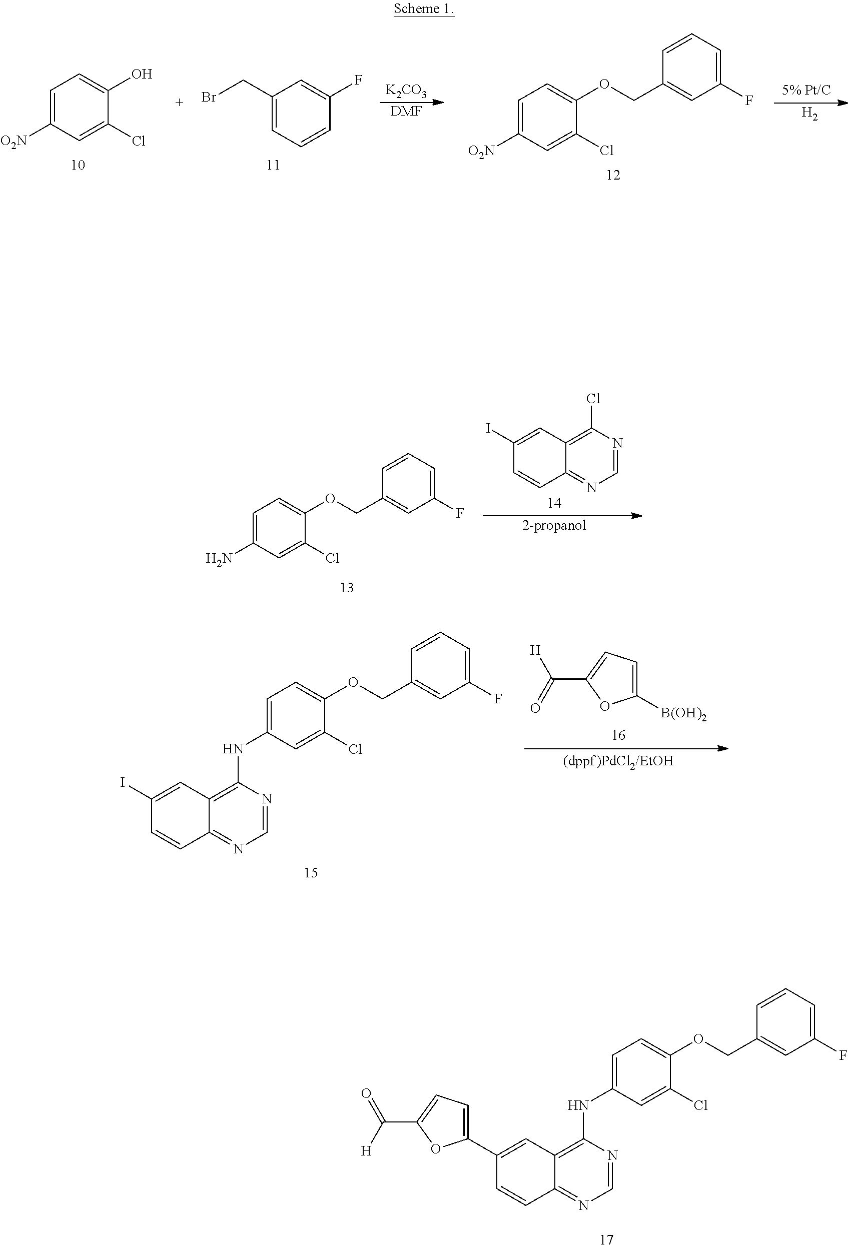

Scheme 1 depicts the synthesis of a certain intermediate useful for the preparation of compounds of the invention wherein Y^{3a}, Y^{4a} and Y^{4b} are all hydrogen. The syntheses of Scheme 1 are further described below.

2-Chloro-1-(3-fluorobenzyloxy-4-nitrobenzene) (12). Powdered potassium carbonate (73.1 g, 0.5300 mol, 1.3 equiv) was added slowly to a solution of 2-chloro-4-nitrophenol (10) (77.6 g, 0.4484 mol, 1.1 equiv) in DMF (300 mL). A thick yellow suspension formed and the reaction temperature increased from 23 to 42° C. The reaction mixture was heated to 80° C. and 3-fluorobenzylbromide (11) (77.1 g, 50 mL, 0.4077 mol, 1.0 equiv) was added dropwise over about 0.5 hr at 80-85° C. using DMF (25 mL) to rinse the addition funnel. The thick suspension was heated at about 95° for 4.5 hr. The reaction mixture was cooled to room temperature then to 10° C. H₂O (500 mL) was added dropwise at <20° C. The yellow suspension was further diluted with H₂O (750 mL) and stirred 1 hr. The solids were filtered, washed with H₂O (2×1 L), dried on the filter for 2 hr then air-dried overnight. The solids were washed with 10% toluene/heptane (500 mL) followed by heptane (500 mL), dried on the filter 1 hr then in a vacuum oven at about 40° C. for 7 hr to give 111.3 g (97%) of 12 as a yellowish-white solid that was used without further purification.

3-Chloro-4-(3-fluorobenzyloxy)phenylamine (13). A mixture of 12 (56.2 g, 0.20 mol) and 5% Pt—C (5.0 g, 50% H₂O) and THF (500 mL) was hydrogenated at 30 psi H₂ until uptake of H₂ ceased (about 2.75 hr). The mixture was filtered through a pad of Celite and then the Celite pad was washed with THF (750 mL). The filtrate was concentrated under reduced pressure to a small volume and residual THF was co-evaporated with toluene (300 mL). The mixture was concentrated to a small volume and seeded. When crystallization was complete, residual toluene was co-evaporated with heptane (2×300 mL). The residual solid was triturated with heptane (200 ml), filtered and dried to give 47.6 g (95%) of 13 as a brownish-white solid that was used without further purification.

4-Chloro-6-iodoquinazoline (14). A suspension of 2-amino-5-iodobenzoic acid (101.3 g; 0.3852 mol) and formamide (210 mL) was heated at about 165° C. for 3.75 hr, with a dark brown solution forming at about 100° C. The mixture was cooled to room temperature and the thick suspension diluted with 50% aqueous ethanol (“EtOH”) (500 mL). The solid was filtered, washed with 50% aqueous EtOH (250 mL) and dried on the filter for 0.5 hr. The solid was washed with EtOH/heptane (1:1 v/v, 500 mL) followed by heptane (250 mL). The solid was air dried overnight followed by drying in a vacuum oven at about 40° C. for 7 hr to give 73.9 g (71%) of 6-iodoquinazolin-4-ol as a gray brown solid that was used without further purification.

Oxalyl chloride (11.8 g, 8.1 mL, 92.6 mmol, 2.0 equiv) was added to a suspension of 6-iodoquinazolin-4-ol (12.6 g, 46.2 mmol, 1.0 equiv), DMF (0.5 ml) and 1,2-dichloroethane (300 mL) resulting in the reaction temperature increasing from 21 to 25° C. The mixture was heated at about 75° C. overnight. TLC (50% EtOAc/heptane) of an aliquot quenched with NaHCO₃ showed the reaction to be incomplete. The mixture was cooled to room temperature, oxalyl chloride (2.0 mL, 0.5 equiv) added and the mixture refluxed for 7 hr. The clear dark brown solution was cooled to room temperature and poured very slowly into 10% aqueous Na₂CO₃ solution (500 mL). The aqueous mixture was extracted with EtOAc (500 mL). Most of the aqueous phase was separated and the remaining mixture filtered to remove some insolubles at the interface. The phases of filtrate were separated, the organic phase washed with brine, dried over Na₂SO₄ and concentrated under reduced pressure. The resulting solid was triturated with cold heptane (about 200 mL), filtered and dried to give 11.2 g (84%) of 14 as a light brown solid that was used without further purification.

[3-Chloro-4-(3-fluorobenzyloxy)phenyl](6-iodoquinazolin-4-yl)amine hydrochloride (15). To a suspension of 14 (12.5 g, 43.0 mmol) in 2-propanol (300 mL) was added 13 (11.2 g, 44.3 mmol). The resulting suspension was refluxed 4 hours and then the volatiles were removed under reduced pressure and the crude solid was triturated with hot acetone (400 mL) and dried at 60° C. for 2 hours to give 15 (16.4 g, 70%) as a pale yellow solid.

5-{4-[3-Chloro-4-(3-fluorobenzyloxy)phenylamino]quinazolin-6-yl}furan-2-carbaldehyde (17). To a suspension of 15 (19.4 g, 35.8 mmol) in ethanol (270 mL) was added triethylamine (24.9 mL, 179 mmol) followed by 5-formylfuran-2-boronic acid (16) (10.0 g, 71.6 mmol). The resulting mixture was purged with nitrogen for 20 min and then Pd(dppf)Cl₂—CH₂Cl₂ (1.18 g, 1.43 mmol) was added. The reaction mixture was refluxed for 2 hours and the volatiles were removed under reduced pressure. The crude residue was taken up in water (500 mL). The precipitate was filtered, washed with water, triturated with methanol (200 mL) and dried at 60° C. to give 17 (16.0 g, 94%) as a tan solid.

### Example 2

### Preparation of Compound 100 Tosylate Salt and Lapatinib Tosylate Salt

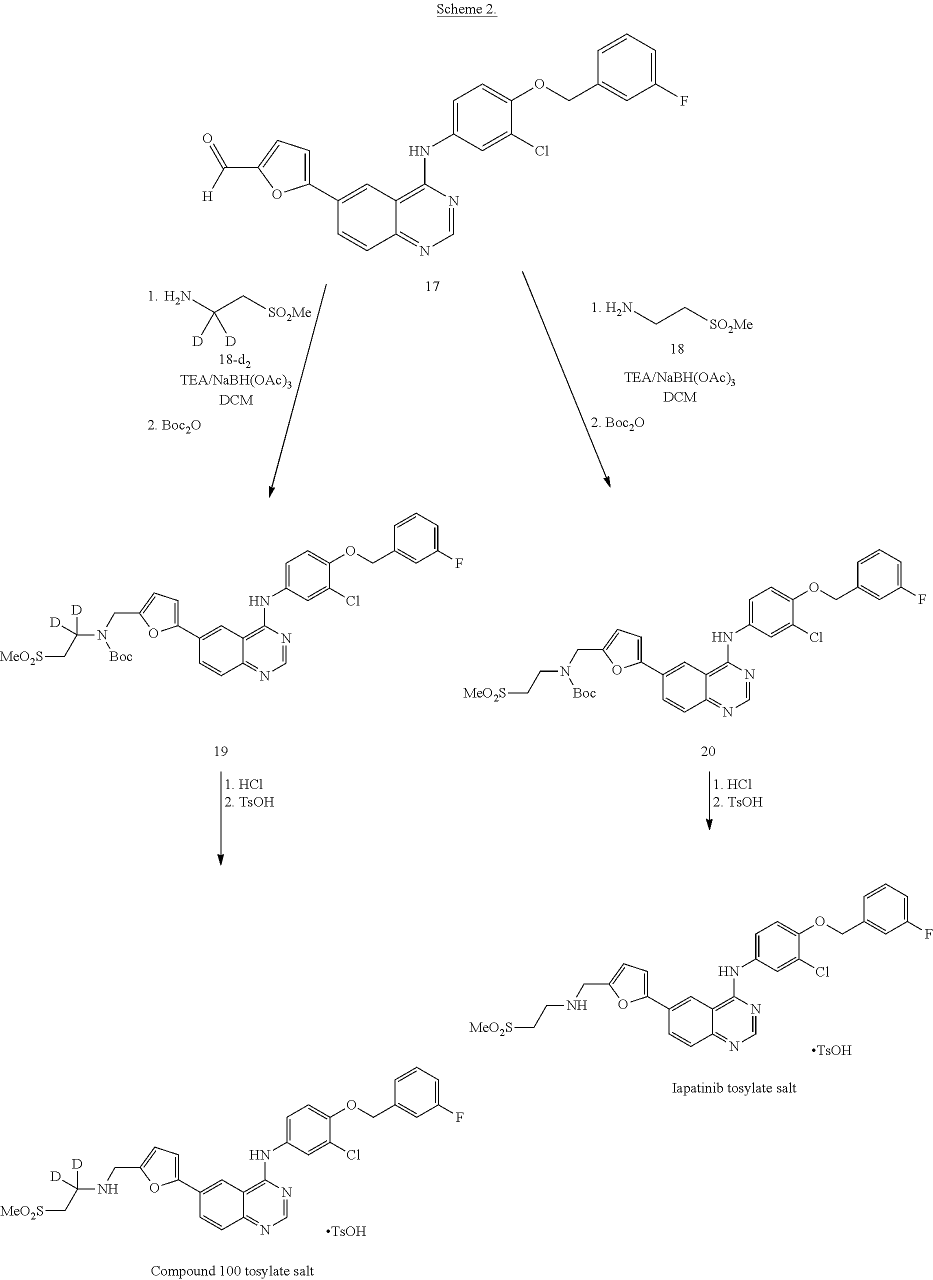

Scheme 2 depicts the synthesis of the tosylate salts of compound 100 and lapatinib. The syntheses of Scheme 2 are further described below.

2-Methanesulfonylethylamine, hydrochloride (18). A mixture of 2-methylsulfanylethylamine (5.0 g, 54.9 mmol, 1.0 equiv), saturated NaHCO₃ solution (100 ml) and THF (200 mL) was cooled to about 13° C. and di-tert-butyl dicarbonate (13.2 g, 60.4 mmol, 1.1 equiv) was added slowly with a slight increase (2° C.) in reaction temperature. The mixture was allowed to warm to room temperature and stirred 3 hr. The mixture was diluted with H₂O (100 mL) and ethyl acetate (“EtOAc”) (200 mL). The organic phase was washed with brine, dried over Na₂SO₄, filtered and concentrated under reduced pressure. The residual pale yellow oil was placed under high vacuum for 1 hr to 12.7 g of crude 2-methanesulfanylethyl)carbamic acid, tert-butyl ester as an oil containing residual t-BuOH and/or Boc₂O that was used without further purification.

70-75% MCPBA (27.0 g, 109.8 mmol) was added portionwise with mild cooling (17-20° C.) to a suspension of crude 2-methanesulfanylethyl)carbamic acid, tert-butyl ester (12.7 g) and NaHCO₃ (10.1 g, 120.8 mmol) in DCM (300 mL). When addition was complete the thick white suspension was stirred at room temperature for 2 hr at which time TLC (EtOAc/heptane, 1:1, v/v) and LCMS showed oxidation was complete. The mixture was diluted with DCM (200 ml) and washed sequentially with 10% aqueous Na₂CO₃ (200 mL), H₂O (200 mL) and brine. The organic phase was dried over Na₂SO₄, filtered and concentrated under reduced pressure to give a pale yellow oil. Seeding was used to induce crystallization. The solid was triturated with cold heptane, filtered and dried to give 10.6 g (86% from 2-methylsulfanylethylamine) of (2-methanesulfonylethyl)carbamic acid, tert-butyl ester as a white solid.

2M HCl in diethyl ether (50 mL) was added to a solution of (2-methanesulfonylethyl)carbamic acid, tert-butyl ester (10.6 g, 47.5 mmol) in EtOAc (250 mL). A precipitate began forming after about 0.25 hr. The suspension was stirred overnight at room temperature. TLC and LCMS showed the reaction was incomplete. 2M HCl in diethyl ether (120 mL) was added and the mixture was stirred overnight at room temperature. The solids were collected, washed with EtOAc (100 mL) and dried under N₂ to give 5.9 g (78%) 18 as a white solid.

2-Methanesulfonyl-1,1-d₂-ethylamine hydrochloride (18-d₂). To a solution of 2-methanesulfonylacetonitrile (5.95 g, 50 mmol) in anhydrous THF (100 mL) was added dropwise a solution of 1.0 M BD₃ in THF (50 mL, 50 mmol) at room temperature. After addition, the reaction was heated overnight at 50° C., cooled to room temperature, and then slowly quenched with methanol (300 mL). The resulting solution was refluxed 3 hours and evaporated in vacuo. The crude residue was taken up in THF (300 mL) and saturated sodium bicarbonate (300 mL) and Boc₂O (10.9 g, 50 mmol) was added. The resulting solution was stirred overnight and extracted with EtOAc (3×300 mL). The combined organic layers were dried over sodium sulfate and evaporated in vacuo to give a viscous oil (13 g). The crude oil was dissolved in 1,4-dioxane (100 mL) and a 4.0 M hydrogen chloride solution in 1,4-dioxane (100 mL) was added. The solution was stirred at room temperature for 2 hours, evaporated in vacuo and chased with methanol to give 2-methanesulfonyl-1,1-d₂-ethylamine hydrochloride as a white solid (4.7 g, 75%) that was used without further purification.

5-{4-[3-Chloro-4-(3-fluorobenzyloxy)phenylamino]quinazolin-6-yl}furan-2-ylmethyl-(2-methanesulfonylethyl)carbamic acid, tert-butyl ester (20). Triethylamine (4.4 mL, 31.8 mmol) was added to a suspension of 18 (4.0 g, 25.05 mmol) in DCM (500 ml) and the mixture stirred for 1 hr at room temperature. 17 (7.1 g, 15 mmol) was added and the suspension was stirred at room temperature for 1 hr to give a clear yellow-brown solution. NaBH(OAc)₃ (9.7 g, 50.1 mmol) was added and the resulting suspension was stirred overnight then quenched by the slow addition of aqueous 10% Na₂CO₃ (300 mL). After 30 min the aqueous phase was separated and the aqueous layer was extracted with ethyl acetate (3×200 mL). The combined organic layers were dried over Na₂SO₄, filtered, and concentrated under reduced pressure to give a brown oil. The crude oil was taken up in THF (300 mL) and saturated sodium bicarbonate and Boc₂O (6.6 g, 30 mmol) was added. The resulting solution was stirred at room temperature for 2 hours and extracted with ethyl acetate (3×500 mL). The combined organic layers were dried over sodium sulfate and evaporated in vacuo to give a crude oil that was purified on a silica-gel column with 3:1 EtOAc/heptane as eluent to give compound 20 (7.0 g, 69%) as a tan foam.

[3-Chloro-4-(3-fluorobenzyloxy)phenyl](6-{5-[(2-methanesulfonylethylamino)-methyl]furan-2-yl}-quinazolin-4-yl)amine 4-toluenesulfonate salt (lapatinib tosylate salt). To a solution of 20 (7.0 g, 10.3 mmol) in DCM (240 mL) in a water bath was added TFA (20 mL). The reaction mixture was stirred overnight at room temperature after which time the volatiles were removed under reduced pressure to give a viscous oil that was neutralized with saturated sodium bicarbonate (200 mL). The resulting suspension was extracted with ethyl acetate (3×300 mL). The combined organic layers were dried over sodium sulfate and evaporated in vacuo to give a tan solid (6 g). The solid was dissolved in absolute ethanol (300 mL) at 65° C. and a solution of p-toluenesulfonic acid monohydrate (1.84 g, 9.7 mmol) in ethanol (25 mL) was added dropwise at this temperature. The resulting suspension was stirred under reflux conditions for 1 hour. The suspension was cooled back to room temperature, filtered and washed with small amount of ethanol. The collected solid was dried overnight at 70° C. to give lapatinib tosylate salt (6.77 g, 93%) as a pale yellow solid.

¹H-NMR (300 MHz, DMSO-d₆): δ 2.29 (s, 3H), 3.14 (s, 3H), 3.41-3.58 (m, 4H), 4.41 (s, 2H), 5.28 (s, 2H), 6.85 (d, J=3.5 Hz, 1H), 7.10 (dd, J₁=7.9 Hz, J₂=0.59 Hz, 2H), 7.16-7.28 (m, 2H), 7.31-7.35 (m, 3H), 7.45-7.50 (m, 3H), 7.73 (dd, J₁=8.8 Hz, J₂=2.3 Hz, 1H), 7.87 (d, J=8.8 Hz, 1H), 8.00 (d, J=2.3 Hz, 1H), 8.24 (dd, J₁=8.8 Hz, J₂=1.8 Hz, 1H), 8.61 (s, 1H), 8.85 (s, 1H), 10.00 (bs, 1H). ¹³C-NMR (75 MHz, DMSO-d₆): δ 21.62, 41.62, 43.76, 50.68, 70.18, 108.79, 114.57, 114.86, 115.02, 115.27, 115.54, 115.89, 118.31, 121.79, 123.37, 123.98, 124.02, 125.16, 126.14, 128.42, 128.72, 129.48, 131.19, 131.29, 133.34, 138.32, 140.20, 140.30, 146.20, 150.64, 154.01, 154.97, 158.39, 161.19, 164.41. ¹⁹F-NMR (282 MHz, DMSO-d₆): δ −113.27. Retention Time (HPLC, method: 20 mm C18-RP column—gradient method 2-95% ACN+0.1% formic acid in 3.3 min with 1.7 min hold at 95% ACN): 2.71 min. MS (M+H⁺): 581.1. Elemental Analysis (C₃₆H₃₄ClFN₄O₇S₂): Calculated: C=57.40, H=4.55, Cl=4.71, F=2.52, N=7.44, S=8.51. Found: C=57.24, H=4.47, Cl=4.92, F=2.62, N=7.40, S=8.53.

(5-{4-[3-Chloro-4-(3-fluorobenzyloxy)phenylamino]quinazolin-6-yl}furan-2-ylmethyl)-(2-methanesulfonyl-1,1-d₂-ethyl)carbamic acid, tert-butyl ester (19). Triethylamine (4.4 mL, 31.8 mmol) was added to a suspension of 18-d₂ (4.05 g, 25.05 mmol) in DCM (500 ml) and the mixture stirred for 1 hr at room temperature. 17 (7.1 g, 15 mmol) was added and the suspension was stirred at room temperature for 1 hr to give a clear brown solution. To this solution was added NaBH(OAc)₃ (9.7 g, 50.1 mmol). The suspension was stirred overnight and quenched by the slow addition of aqueous 10% Na₂CO₃ (300 mL). After 30 min Boc₂O (6.6 g, 30 mmol) was added. The resulting solution was stirred at room temperature for 2 hours. The layers were separated and the aqueous layer was extracted with ethyl acetate (3×500 mL). The combined organic layers were dried over sodium sulfate and evaporated in vacuo to give a crude oil that was purified on a silica-gel column with 3:1 EtOAc/heptane as eluent to give 19 (6.0 g, 59%) as a tan foam.

[3-Chloro-4-(3-fluorobenzyloxy)phenyl](6-{5-[(2-methanesulfonyl-1,1-d₂-ethylamino)methyl]furan-2-yl}-quinazolin-4-yl)amine, 4-toluenesulfonate salt (Compound 100 tosylate salt). To a solution of 19 (6.0 g, 8.8 mmol) in DCM (240 mL) in a water bath was added TFA (20 mL). The reaction mixture was stirred overnight at room temperature after which time the volatiles were removed under reduced pressure to give a viscous oil that was neutralized with saturated sodium bicarbonate (300 mL). The resulting suspension was extracted with ethyl acetate (3×300 mL). The combined organic layers were dried over sodium sulfate and evaporated in vacuo to give a tan solid (5.1 g). The solid was dissolved in absolute ethanol (300 mL) at 65° C. and a solution of p-toluenesulfonic acid monohydrate (1.56 g, 8.22 mmol) in ethanol (25 mL) was added at this temperature. The resulting suspension was stirred under reflux conditions for 1 hour. The suspension was cooled back to room temperature, filtered, and washed with small amount of ethanol. The collected solid was dried overnight at 70° C. to give Compound 100 tosylate salt (5.32 g, 80%) as a yellow solid.

¹H-NMR (300 MHz, DMSO-d₆): δ 2.29 (s, 3H), 3.14 (s, 3H), 3.54 (s, 2H), 4.41 (s, 2H), 5.28 (s, 2H), 6.85 (d, J=3.2 Hz, 1H), 7.09-7.20 (m, 4H), 7.28-7.35 (m, 3H), 7.47-7.49 (m, 3H), 7.73 (dd, J1=8.8 Hz, J2=2.0 Hz, 1H), 7.87 (d, J=8.5 Hz, 1H), 7.99 (d, J=2.1 Hz, 1H), 8.23 (d, J=8.8 Hz, 1H), 8.61 (s, 1H), 8.85 (s, 1H), 10.00 (bs, 1H). ¹³C-NMR (75 MHz, DMSO-d₆): δ 21.62, 41.63, 43.70, 50.50, 70.16, 108.79, 114.57, 114.87, 115.02, 115.27, 115.55, 115.90, 118.32, 121.79, 123.36, 123.99, 124.02, 125.16, 126.14, 128.41, 128.71, 129.46, 131.19, 131.30, 133.34, 138.27, 140.20, 146.27, 150.65, 154.02, 155.00, 158.40, 164.41. ¹⁹F-NMR (282 MHz, DMSO-d₆): δ −113.27. Retention Time (HPLC, method: 20 mm C18-RP column—gradient method 2-95% ACN+0.1% formic acid in 3.3 min with 1.7 min hold at 95% ACN): 2.71 min. MS (M+H⁺): 582.9. Elemental Analysis (C₃₆H₃₂D₂ClFN₄O₇S₂): Calculated: C=57.25, H=4.80, Cl=4.69, F=2.52, N=7.42, S=8.49. Found: C=56.87, H=4.30, Cl=5.47, F=2.54, N=7.31, S=8.49.

### Example 3

### Synthesis of Tributylstannyl Reagent 24

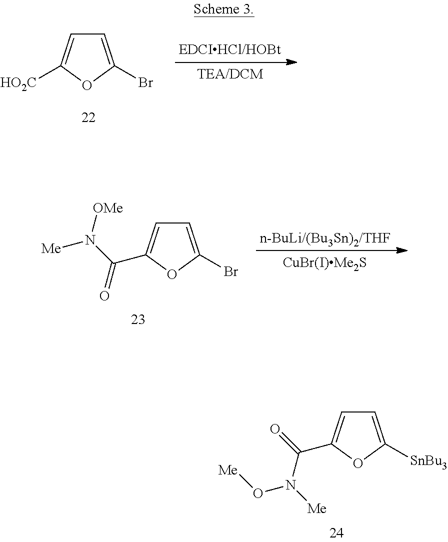

Scheme 3 depicts the synthesis of a tributylstannyl reagent used in the synthesis of compounds of the present invention. The syntheses of Scheme 3 are further described below.

5-Bromo-furan-2-carboxylic acid methoxy-methyl-amide (23). As shown in Scheme 4, to a suspension of 1-ethyl-3-(3′-dimethylaminopropyl)carbodiimide (“EDCI”).HCl (75.0 g, 391.6 mmol) in dichloromethane (“DCM”) (800 mL) in an ice/water bath was added triethylamine (124.8 mL, 890.0 mmol). Five minutes later 5-bromo-2-furoic acid (22) (68 g, 356.0 mmol) and anhydrous HOBt (52.9 g, 391.6 mmol) were added. The reaction mixture was stirred another 10 min in the ice/water bath and O-methyl-n-methylhydroxylamine hydrochloride (38.2 g, 391.6 mmol) was added. The reaction was allowed to warm to room temperature overnight. The reaction was quenched with water (1.5 L) and the layers were split. The aqueous layer was extracted with DCM (2×500 mL) and the combined organic layers were dried over sodium sulfate and evaporated in vacuo to give a crude oil that was purified on a Silica-gel column with 1:4 EtOAc/heptane as eluent to give compound 23 (78 g, 85%) as a pale yellow oil.

5-Tributylstannyl-furan-2-carboxylic acid methoxy-methyl-amide (24). To a solution of bis(tributyltin) (200 g, 344.8 mmol) in anhydrous THF (450 mL) at −20° C. was added nBuLi (1.6 M in hexanes, 210.6 mL, 336.9 mmol) over a period of 20 min. Then the reaction mixture was cooled to −50° C. and copper(I) bromide dimethyl sulfide complex (34.6 g, 168.5 mmol) was added. The reaction mixture was warmed back to −40° C. and stayed at this temperature for 20 min. Then the reaction mixture was cooled to −78° C. and a solution of 23 (26.3 g, 112.3 mmol) in THF (150 mL) was added. The reaction was stirred for 3 hours at −78° C. and then for 1 hour at −40° C. The cooling bath was removed and the reaction was quenched with 20 wt % ammonium chloride (1.5 L) and diluted with MTBE (1.0 L). 15 minutes later the layers were split and the aqueous layer was extracted with MTBE (2×1.0 L). The combined organic layers were dried over sodium sulfate and evaporated in vacuo. The crude residue was taken up with 1:1 MTBE/heptane and directly loaded onto a silica gel column (2 kg) and eluted with 1:1 MTBE/heptane to afford compound 24 (34.0 g, 68%) as a pale yellow oil.

### Example 4

### Preparation of Mono-Deuterated Intermediate 17

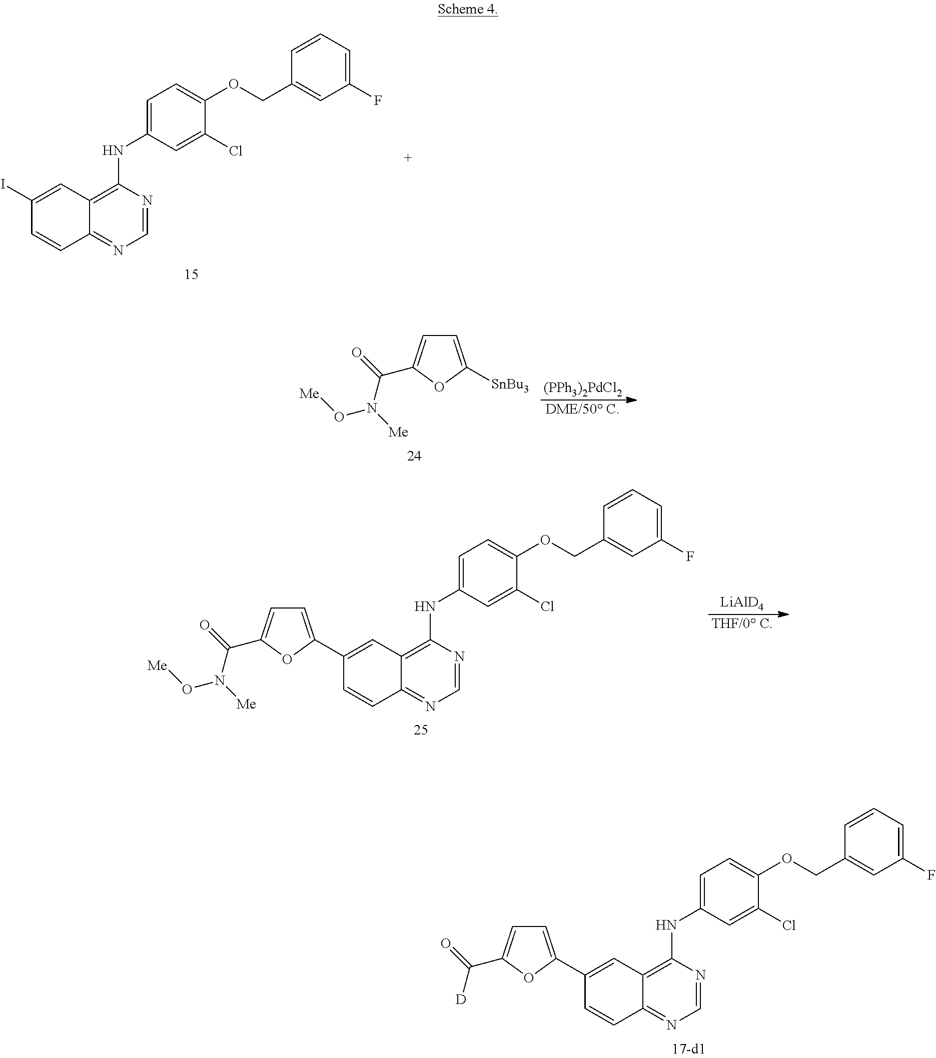

Scheme 4 depicts the synthesis of a certain intermediate useful for the preparation of compounds of the invention wherein Y^{3a} is deuterium; and Y^{4a} and Y^{4b} are both hydrogen. The syntheses of Scheme 4 are further described below.

5-{4-[3-Chloro-4-(3-fluoro-benzyloxy)-phenylamino]-quinazolin-6-yl}-furan-2-carboxylic acid methoxy-methyl-amide (25). To a suspension of 15 (27.0 g, 53.3 mmol) in 1,2-dimethoxyethane (700 mL) at room temperature was added triethylamine (7.5 mL, 53.3 mmol). The reaction mixture was stirred 10 min and purged with nitrogen for 30 min. To the solution formed above was added 24 (34.0 g, 76.5 mmol) followed by (PPh₃)₂PdCl₂ (2.7 g). The reaction was heated at 50° C. until reaction was complete (ca. 24-48 hours). Upon the completion of the reaction it was evaporated under vacuum to remove 80% of the solvent and diluted with MTBE (500 mL). The precipitate was filtered, washed with MTBE (500 mL) and water (500 mL), and dried at 50° C. overnight to afford compound 25 (22.0 g, 77%) as a tan solid. A small sample was purified on a silica-gel column with 1:1 to 4:1 EtOAc/heptane as eluent to afford 25 as a white solid.

5-{4-[3-Chloro-4-(3-fluorobenzyloxy)phenylamino]quinazolin-6-yl}furan-2-carbaldehyde-d (17-d1). To a solution of 25 (22.0 g, 41.3 mmol) in THF in an ice/water/salt bath was added lithium aluminum deuteride (1.73 g, 41.3 mmol) in portions while the internal temperature was maintained below 5° C. The reaction was stirred for 1 hour in the cooling bath, quenched with deuterium oxide (20 mL), diluted with ethyl acetate (1.0 L), dried over sodium sulfate, filtered, and evaporated in vacuo to give the title compound (17-d1) as a tan solid in quantitative yield.

### Example 5

### Synthesis of Hepta-Deuterated Amine Reagent 30

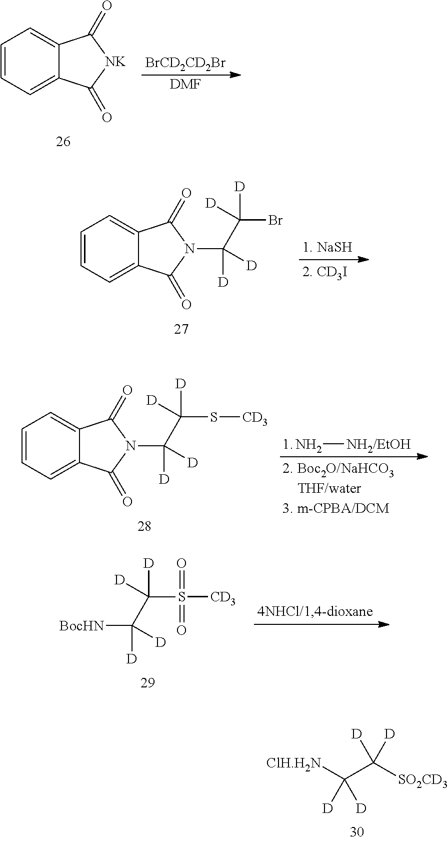

Scheme 5 depicts the synthesis of a heptadeuterated amine reagent used in the synthesis of compounds of the present invention wherein Y^{1a}, Y^{1b}, Y^{1c}, Y^{2a}, Y^{2b}, Y^{5a} and Y^{5b} are simultaneously deuterium. The syntheses of Scheme 5 are further described below.

2-(2-Bromo-1,1,2,2-d₄-ethyl)-isoindole-1,3-dione (27). To a solution of 1,2-dibromoethane-d₄ (100 g, 521.1 mmol) in anhydrous DMF (580 mL) at ambient temperature was added phthalimide potassium salt (26) (48.3 g, 260.6 mmol). The resulting mixture was stirred at room temperature for 48 hours, filtered, and washed with small amount of DMF. The filtrate was diluted with MTBE (1.6 L) and washed with water (1.4 L). The aqueous layer was extracted with MTBE (2×1.2 L). The combined organic layers were washed with water (2×1.0 L), dried over sodium sulfate, and evaporated in vacuo to afford a crude white solid that was triturated with heptane (600 mL) to give compound 27 (105 g, containing bis alkylation product) as a white solid.

2-(2-d₃-Methylsulfanyl-1,1,2,2-d₄-ethyl)-isoindole-1,3-dione (28). To a solution of 27 (66.8 g, 258.7 mmol) in DMF (360 mL) at 0° C. was added sodium hydrogen sulfide hydrate (23.0 g, 310 mmol). The reaction mixture was stirred for 20 min at 0° C. and for 1 hour at ambient temperature. To the reaction mixture formed above in a water bath was added potassium carbonate (47.6 g, 344.9 mmol) followed by iodomethane-d₃ (50 g, 344.9 mmol). The reaction was stirred overnight at room temperature, quenched with water (1.5 L), and extracted with MTBE (3×1.0L). The combine organic layers were washed with brine (1.5 L) and water (1.5 L), dried over sodium sulfate, and evaporated in vacuo to give a crude solid that was purified on an silica gel column with 1:4 MTBE/heptane as eluent to give compound 28 (39.72 g, 67%) as a white solid.

(2-d₃-Methanesulfonyl-1,1,2,2-d₄-ethyl)-carbamic acid tert-butyl ester (29). To a solution of 28 (39.7 g, 174.0 mmol) in ethanol (1.3 L) was added hydrazine monohydrate (10.4 g, 208.8 mmol). The reaction was stirred under reflux conditions overnight, cooled to room temperature, diluted with ethyl ether (1.5 L), filtered, and washed with ethyl ether (500 mL). The filtrate was evaporated in vacuo at 30° C. to give a clear oil. The oil was taken up with THF/water (300 mL/300 mL) and Boc₂O (45.6 g, 208.8 mmol) was added. The reaction mixture was stirred for 2 hours at room temperature, and extracted with ethyl acetate (3×300 mL). The combined organic layers were dried over sodium sulfate and evaporated in vacuo to give a crude oil that was purified on a silica gel column with 1:4 MTBE/heptane as eluent to afford a clear oil (20.0 g). The oil (20 g, 101.0 mmol) was dissolved in DCM (575 mL) in a water bath and sodium bicarbonate (19.3 g, 230.0 mmol) was added. 3-chloroperoxybenzoic acid (41.5 g, 201.5 mmol) was added in portions. The reaction mixture was stirred for 2 hours at room temperature and diluted with DCM (1.7 L) and water (1.7 L). The layers were split and the aqueous layer was extracted with DCM (1 L). The combined organic layers were washed with 10 wt % potassium carbonate (1.0 L) and water (1.0 L), dried over sodium sulfate, and evaporated in vacuo to give a solid that was triturated with heptane (400 mL) to give compound 29 (25.3 g, 63%) as a crystalline white solid.

2-d₃-Methanesulfonyl-1,1,2,2-4-ethylamine hydrochloride (30). To a solution of 29 (13.0 g, 56.2 mmol) in 1,4-dioxane (50 mL) was added 4.0 M HCl in 1,4-dioxane (250 mL). The reaction mixture was stirred overnight at room temperature and evaporated in vacuo to afford compound 30 as a white solid in quantitative yield.

### Example 6

### Synthesis of Compound 101 Tosylate Salt

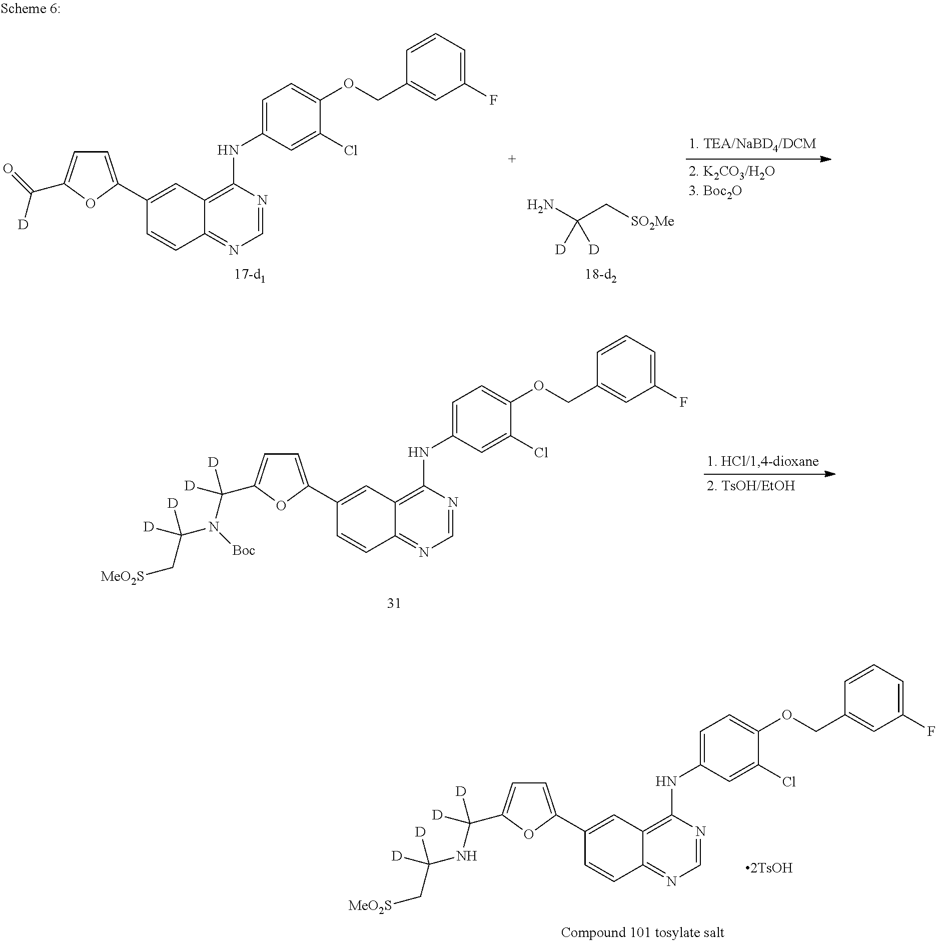

Scheme 6 depicts the synthesis of Compound 101 tosylate salt. The syntheses of Scheme 6 are further described below.

(5-{4-[3-Chloro-4-(3-fluorobenzyloxy)phenylamino]quinazolin-6-yl}furan-2-yl-methyl-d₂)-(2-methanesulfonyl-1,1-d₂-ethyl)carbamic acid, tert-butyl ester (31). To a suspension of 18-d2 obtained above (4.7 g, 37.5 mmol) in DCM (400 mL) was added triethylamine (8.0 mL, 50 mmol). 10 minutes later 17-d1 (6.4 g, 13.5 mmol) and sodium sulfate (20 g) were added. After reaction mixture was stirred 3 hours at room temperature, sodium borodeuteride (1.88 g, 45.8 mmol) was added in portions. The resulting mixture was stirred overnight at room temperature and quenched with 10 wt % potassium carbonate in deuterium oxide (200 mL). 20 min later Boc₂O (10.9 g, 50.0 mmol) was added and the reaction was stirred at room temperature for 2 hours. The layers were split and the aqueous layer was extracted with ethyl acetate (2×300 mL). The combined organic layers were dried over sodium sulfate and evaporated in vacuo to afford a crude residue that was purified on a silica-gel column with ethyl acetate as eluent to afford compound 31 (6.7 g, 72%) as a viscous oil.

[3-Chloro-4-(3-fluorobenzyloxy)phenyl]-(6-{5-[(2-methanesulfonyl-1,1-d₂-ethylamino)-d₂-methyl]-furan-2-yl}-quinazolin-4-yl)amine di-tosylate (Compound 101 tosylate salt). To a solution of 31 (6.7 g, 9.77 mmol) in 1,4-dioxane (40 mL) at room temperature was added 4.0 M HCl in 1,4-dioxane (200 mL). The reaction mixture was stirred for 3 hours at room temperature then evaporated in vacuo. The resulting yellow solid was suspended in ethyl acetate (300 mL) and neutralized with 10 wt % potassium carbonate in deuterium oxide (100 mL). The layers were separated and the aqueous layer was extracted with ethyl acetate (2×300 mL). The combined organic layers were dried over sodium sulfate and evaporated in vacuo to give a tan foam (3.5 g, 5.99 mmol). The tan foam was dissolved in THF (15 mL) and added to a solution of p-toluenesulfonate monohydrate (2.85 g, 15.0 mmol) in absolute ethanol (50 mL) at 60° C. The suspension was refluxed for 1 hour and cooled to room temperature. The precipitate was collected by suction filtration, washed with small amount of absolute ethanol, then dried at 40° C. for 4 hours to afford the title compound (Compound 101 tosylate salt) (3.6 g) as a yellow solid.

¹H-NMR (300 MHz, DMSO-d₆): δ 2.28 (s, 6H), 3.13 (s, 3H), 3.57 (s, 2H), 5.32 (s, 2H), 6.90 (d, J=3.5 Hz, 1H), 7.10 (d, J=7.9 Hz, 4H), 7.18-7.27 (m, 2H), 7.31-7.36 (m, 3H), 7.49 (d, J=7.9, 4H), 7.49 (m, 1H), 7.62 (dd, J₁=8.9 Hz, J₂=2.5 Hz, 1H), 7.87 (d, J=2.6 Hz, 1H), 7.93 (d, J=8.8 Hz, 1H), 8.43 (d, J=8.9 Hz, 1H), 8.94 (s, 1H), 9.05 (s, 1H), 9.25 (bs, 1H), 11.40 (bs, 1H). ¹³C-NMR (75 MHz, DMSO-d₆): δ 21.46, 41.37, 50.06, 70.03, 110.15, 114.62, 114.72, 114.84, 114.91, 115.38, 115.67, 115.83, 119.14, 121.93, 124.05, 124.08, 125.35, 126.14, 127.08, 128.82, 130.33, 130.78, 131.30, 131.40, 132.06, 138.60, 140.00, 140.09, 145.88, 147.28, 152.55, 153.04, 160.21, 161.27, 164.51. ¹⁹F-NMR (282 MHz, DMSO-d₆): δ −113.37. Retention Time (HPLC, method: 20 mm C18-RP column—gradient method 2-95% ACN+0.1% formic acid in 3.3 min with 1.7 min hold at 95% ACN): 2.72 min. MS (M+H⁺): 585.3. Elemental Analysis (C₄₃H₃₈D₄ClFN₄O₁₀S₃): Calculated: C=55.56, H=4.55, Cl=3.81, F=2.04, N=6.03, S=10.35. Found: C=55.61, H=4.45, Cl=4.22, F=2.14, N=5.92, S=10.37.

### Example 7

### Synthesis of Compound 102 Tosylate Salt

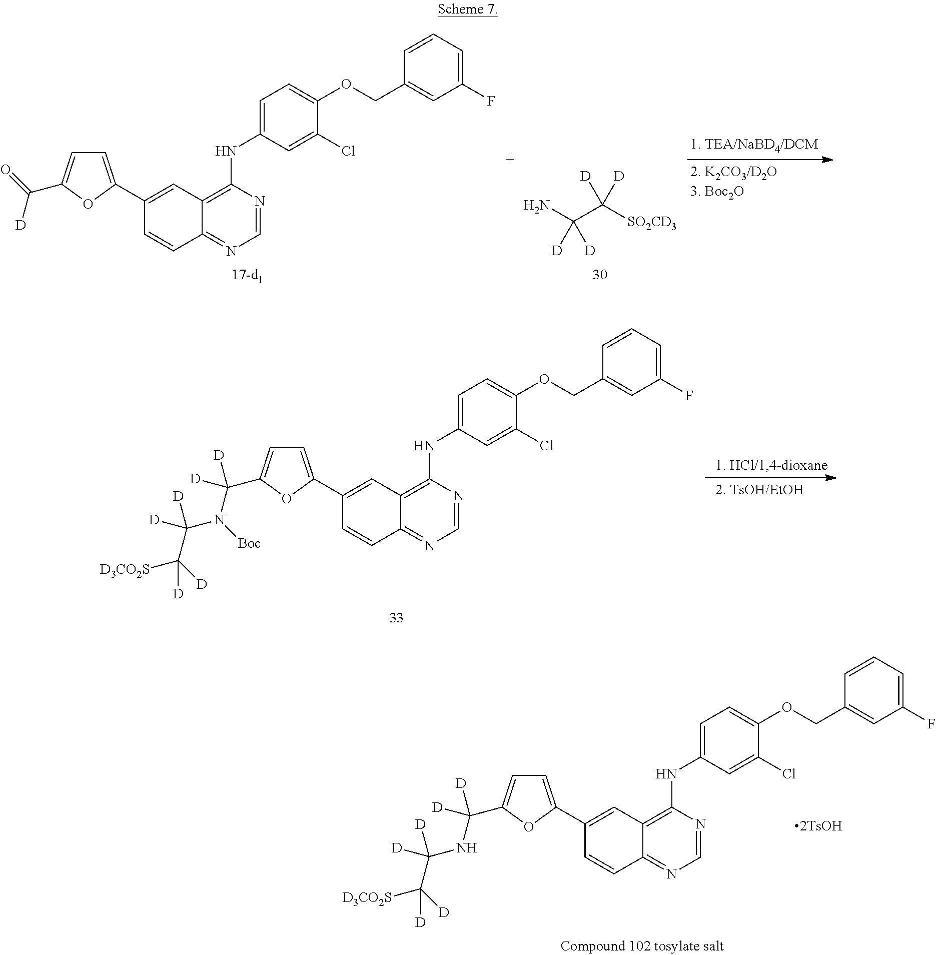

Scheme 7 depicts the synthesis of Compound 102 tosylate salt. The syntheses of Scheme 7 are further described below.

(5-{4-[3-Chloro-4-(3-fluorobenzyloxy)phenylamino]quinazolin-6-yl}furan-2-ylmethyl-d₂-)-(2-d₃-methanesulfonyl-1,1,2,2-dᵣethyl)carbamic acid, tert-butyl ester (33). To a suspension of 30 (ca. 65.0 mmol) in DCM (850 mL) was added triethylamine (9.1 mL, 65 mmol). 10 minutes later 17-d1 (12.0 g, 25.2 mmol) and sodium sulfate (20 g) were added. After the reaction mixture was stirred for 3 hours at room temperature, sodium borodeuteride (3.5 g, 85.7 mmol) was added in portions. The resulting mixture was stirred overnight at room temperature and quenched with 10 wt % potassium carbonate in deuterium oxide (300 mL). 20 min later Boc₂O (14.2 g, 65.0 mmol) was added and the reaction was stirred at room temperature for 2 hours. The layers were split and the aqueous layer was extracted with ethyl acetate (2×400 mL). The combined organic layers were dried over sodium sulfate and evaporated in vacuo to afford a crude residue that was purified on a silica-gel column with ethyl acetate as eluent to afford compound 33 (8.0 g, 46%) as an orange foam.

[3-Chloro-4-(3-fluoro-benzyloxy)-phenyl]-(6-{5-[(2-d₃-methanesulfonyl-1,1,2,2-4-ethylamino)-d₂-methyl]-furan-2-yl}-quinazolin-4-yl)amine di-tosylate (Compound 102 tosylate salt). To a solution of 33 (8.0 g, 11.6 mmol) in 1,4-dioxane (50 mL) at room temperature was added 4.0 M HCl in 1,4-dioxane (300 mL). The reaction mixture was stirred for 3 hours at room temperature and evaporated in vacuo. The yellow solid was suspended in ethyl acetate (400 mL) and neutralized with 10 wt % potassium carbonate in deuterium oxide (200 mL). The layers were split and the aqueous layer was extracted with ethyl acetate (2×300 mL). The combined organic layers were dried over sodium sulfate and evaporated in vacuo to give an orange solid (ca. 11.6 mmol). The solid was dissolved in THF (40 mL) and added to a solution of p-toluenesulfonate monohydrate (5.5 g, 29.0 mmol) in absolute ethanol (150 mL) at 60° C. The suspension was stirred under reflux conditions for 1 hour then was cooled to room temperature. The precipitate was collected by suction filtration, washed with a small amount of absolute ethanol, then dried at 40° C. for 4 hours to afford the title compound (Compound 102 tosylate salt) (7.9 g) as a yellow solid.

¹H-NMR (300 MHz, DMSO-d₆): δ 2.28 (s, 6H), 5.31 (s, 2H), 6.90 (d, J=3.5 Hz, 1H), 7.10 (d, J=7.9 Hz, 4H), 7.18-7.25 (m, 2H), 7.31-7.36 (m, 3H), 7.48 (d, J=8.2, 4H), 7.48 (m, 1H)7.62 (dd, J₁=9.1 Hz, J₂=2.3 Hz, 1H), 7.88 (d, J=2.6 Hz, 1H), 7.93 (d, J=8.8 Hz, 1H), 8.41 (dd, J₁=8.8 Hz, J₂=1.4 Hz, 1H), 8.92 (s, 1H), 9.03 (s, 1H), 9.28 (bs, 1H), 11.32 (bs, 1H). ¹³C-NMR (75 MHz, DMSO-d₆): δ 21.45, 70.13, 110.11, 114.59, 114.76, 114.89, 114.93, 115.36, 115.63, 115.80, 119.16, 122.01, 124.02, 124.05, 125.29, 126.15, 127.02, 128.79, 130.32, 130.91, 131.26, 131.38, 132.03, 138.54, 140.02, 140.12, 145.98, 147.26, 151.97, 152.55, 153.11, 160.18, 161.30, 164.53. ¹⁹F-NMR (282 MHz, DMSO-d₆): δ −113.40. Retention Time (HPLC, method: 20 mm C18-RP column—gradient method 2-95% ACN+0.1% formic acid in 3.3 min with 1.7 min hold at 95% ACN): 2.74 min. MS (M+H⁺): 590.1. Elemental Analysis (C₄₃H₃₃D₉ClFN₄O₁₀S₃): Calculated: C=55.27, H=4.53, Cl=3.79, F=2.03, N=6.03, S=10.29. Found: C=55.28, H=4.56, Cl=3.90, F=2.00, N=6.00, S=10.16.

### Example 8

### Synthesis of Compound 103 Tosylate Salt

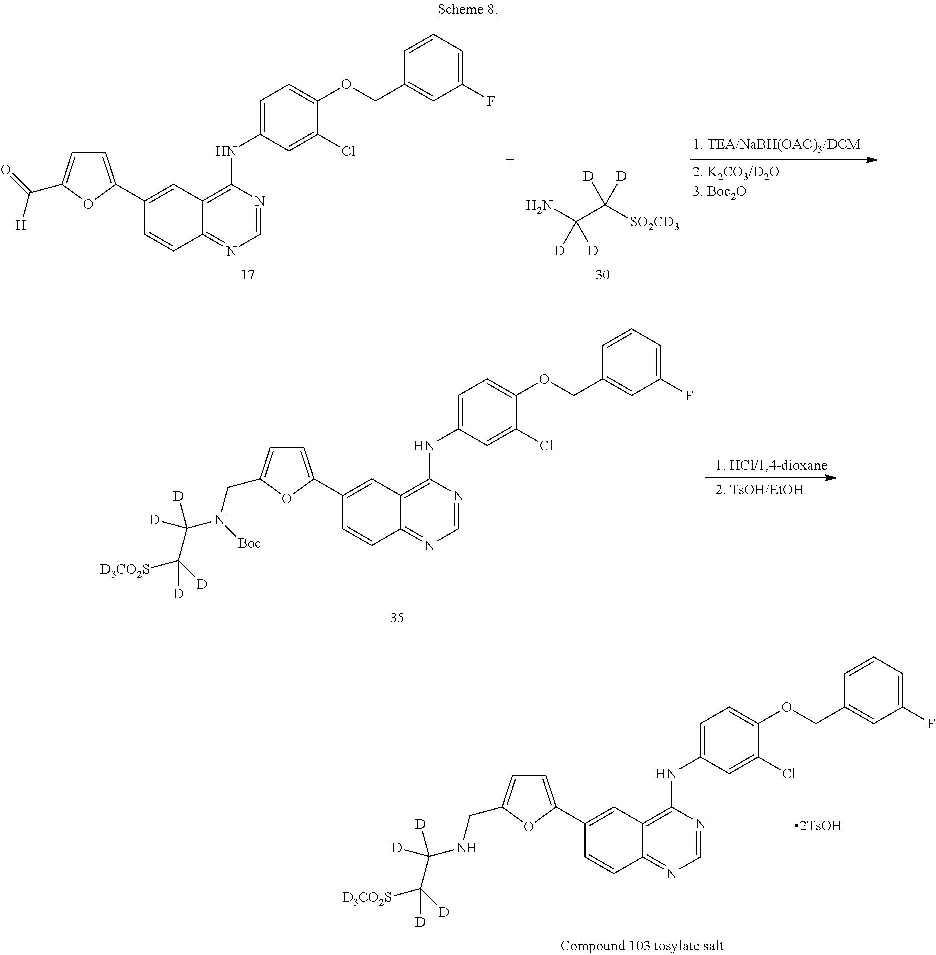

Scheme 8 depicts the synthesis of a compound 103 tosylate salt. The syntheses of Scheme 8 are further described below.

(5-{4-[3-Chloro-4-(3-fluorobenzyloxy)phenylamino]quinazolin-6-yl}furan-2-ylmethyl)-(2-d₃-methanesulfonyl-1,1,2,2-4-ethyl)carbamic acid, tert-butyl ester (35). To a suspension of 30 (ca. 56.2 mmol) in DCM (850 mL) was added triethylamine (9.1 mL, 65 mmol). 10 minutes later 17 (13.0 g, 27.4 mmol) was added. After the reaction mixture was stirred 1 hour at room temperature, sodium triacetoxyborohydride (17.8 g, 91.5 mmol) was added in portions. The resulting mixture was stirred overnight at room temperature and quenched with 10 wt % potassium carbonate in deuterium oxide (300 mL). 20 min later Boc₂O (14.2 g, 65.0 mmol) was added and the reaction was stirred at room temperature for 2 hours. The layers were split and the aqueous layer was extracted with ethyl acetate (2×400 mL). The combined organic layers were dried over sodium sulfate and evaporated in vacuo to afford a crude residue that was purified on a silica-gel column with ethyl acetate as the eluent to afford compound 35 (10.5 g, 56%) as an orange foam.

[3-Chloro-4-(3-fluoro-benzyloxy)-phenyl]-(6-{5-[(2-d₃-methanesulfonyl-1,1,2,2-4-ethylamino)-methyl]-furan-2-yl}-quinazolin-4-yl)amine di-tosylate (Compound 103 tosylate salt). To a solution of 35 (10.5 g, 15.3 mmol) in 1,4-dioxane at room temperature was added 4.0 M HCl in 1,4-dioxane (200 mL). The reaction mixture was stirred for 3 hours at room temperature and evaporated in vacuo. The yellow solid was suspended in ethyl acetate (300 mL) and neutralized with 10 wt % potassium carbonate in deuterium oxide (200 mL). The layers were split and the aqueous layer was extracted with ethyl acetate (400 mL). The combined organic layers were dried over sodium sulfate and evaporated in vacuo to give a tan solid (6.0 g, 10.2 mmol). The tan solid was dissolved in THF (20 mL) and added to a solution of p-toluenesulfonate monohydrate (4.9 g, 25.5 mmol) in absolute ethanol (80 mL) at 60° C. The suspension was stirred under reflux conditions for 1 hour then was cooled to room temperature. The precipitate was collected by suction filtration, washed with small amount of absolute ethanol, then dried at 40° C. for 4 hours to afford the title compound (Compound 103 tosylate salt) (4.8 g) as a yellow solid.

¹H-NMR (300 MHz, DMSO-d₆): δ 2.28 (s, 6H), 4.48 (s, 2H), 5.31 (s, 2H), 6.89 (d, J=3.5 Hz, 1H), 7.10 (d, J=7.9 Hz, 4H), 7.17-7.35 (m, 5H), 7.49 (d, J=7.9, 4H), 7.49 (m, 1H), 7.62 (dd, J₁=8.8 Hz, J₂=2.3 Hz, 1H), 7.87 (d, J=2.6 Hz, 1H), 7.93 (d, J=8.8 Hz, 1H), 8.43 (d, J=8.8 Hz, 1H), 8.93 (s, 1H), 9.05 (s, 1H), 9.33 (bs, 1H), 11.38 (bs, 1H). ¹³C-NMR (75 MHz, DMSO-d₆): δ 21.45, 43.34, 70.13, 110.14, 114.59, 114.76, 114.92, 115.35, 115.63, 115.80, 119.14, 122.01, 124.02, 124.05, 125.31, 126.15, 127.04, 128.81, 130.36, 130.90, 131.26, 131.38, 132.08, 138.62, 139.29, 140.03, 140.13, 145.86, 147.33, 151.93, 152.56, 153.10, 160.19, 161.30, 164.53. ¹⁹F-NMR (282 MHz, DMSO-d₆): δ −113.39. Retention Time (HPLC, method: 20 mm C18-RP column—gradient method 2-95% ACN+0.1% formic acid in 3.3 min with 1.7 min hold at 95% ACN): 2.72 min. MS (M+H⁺): 588.3. Elemental Analysis (C₄₃H₃₅D₇ClFN₄O₁₀S₃): Calculated: C=55.38, H=4.54, Cl=3.80, F=2.04, N=6.01, S=10.32. Found: C=55.07, H=4.24, Cl=4.50, F=2.06, N=5.86, S=10.17.

### Example 9

### Synthesis of Di-Deutero Intermediate 40

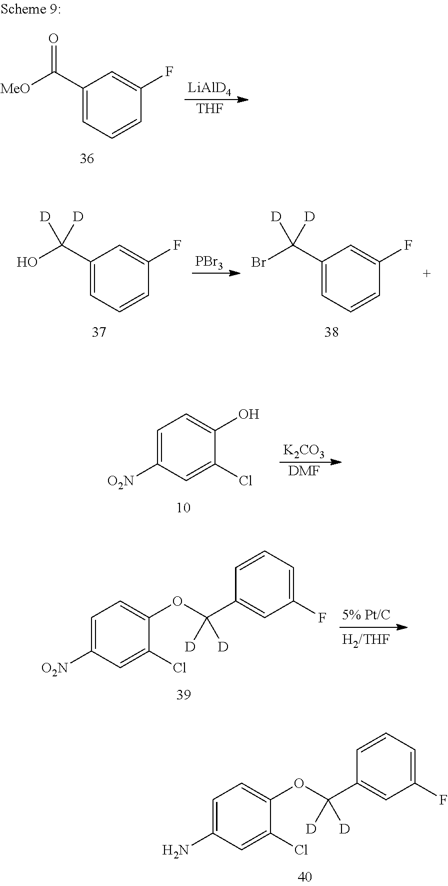

Scheme 9 depicts the synthesis of a common intermediate used in the further synthesis of compounds of this invention wherein Y^{4a} and Y^{4b} are simultaneously deuterium. The synthesis steps of Scheme 9 are described in greater detail below.

3-Fluoro-α,α-d₂-benzyl alcohol (37). To a solution of methyl 3-fluorobenzoate (36; 182 g, 1.181 mol) in THF (2.0 L) at −78° C. was added LiAlD₄ (50 g, 1.181 mol) in portions. The reaction was allowed to warm to room temperature then was stirred overnight at room temperature. Upon completion, the reaction mixture was cooled to 0° C., and then was slowly quenched with water (50 mL), 15 wt % sodium hydroxide (50 mL), and water (50 mL). The resulting mixture was stirred for approximately 2 h, filtered over celite, and the celite washed with THF. The filtrate was rid of solvent in vacuo, dissolved in THF, then reduced in vacuo again to give 37 (125.1 g) as a clear oil in 83% yield.

3-Fluoro-α,α-d₂-benzyl bromide (38). To a solution of 37 (125.1 g, 976.3 mmol) in dichloromethane (1.64 L) at −20° C. was added dropwise phosphorus bromide (165 mL, 1.753 mol). The reaction was stirred for 3 h at −20° C., was warmed to 0° C. and stirred for an additional hour, then was quenched with water (1.5 L), and slowly brought to pH 8 with solid potassium carbonate. The layers were separated and the aqueous layer was extracted with dichloromethane (2×1.0 L). The combined organic layers were dried over sodium sulfate and rid of solvent in vacuo to give 38 (160.5 g, 86%) as a clear oil.

2-Chloro-1-(3-fluoro-α,α-d₂-benzyloxy-4-nitrobenzene (39). To a solution of 2-chloro-4-nitrophenol (10; 160.4 g, 924.1 mmol, 1.1 equiv) in DMF (650 mL) was added powdered potassium carbonate (73.1 g, 0.53 mol, 1.3 equiv) resulting in a mildly exothermic reaction. The resulting thick yellow suspension was stirred and heated to 80° C. prior to dropwise addition of 3-fluoro-α,α-d₂-benzylbromide (38; 160.5 g, 840.1 mmol, 1.0 equiv) over 30 min at 80-85° C. using DMF (25 mL) to rinse the addition funnel. The thick suspension was then heated to ˜95° C. and stirred for 4.5 h before cooling, first to room temperature, then to 10° C., and quenching with water (2.7 L) while maintaining the temperature below 20° C. The suspension was stirred for 1 h at room temperature, the solids were removed by filtration, washed with H₂O (2×2.1 L), dried on the filter paper for 2 h, air-dried overnight, further washed with 10% toluene/heptane (1.1 L) followed by heptane (1.1 L), dried on the filter paper 1 h and then in a vacuum oven at 40° C. overnight to give 39 as a yellow solid in quantitative yield.

3-Chloro-4-(3-fluoro-α,α-d₂-benzyloxy)phenylamine (40). A mixture of 39 (ca. 420 mmol) and 5% Pt—C (12.0 g, containing 50% H₂O) in THF (1.0 L) was submitted to hydrogenation at 30 psi H₂ until the consumption of H₂ ceased (˜2.75 h). The mixture was filtered through a pad of Celite along with the resulting mixture of a duplicate reaction on the same scale and the Celite pad was washed with THF (2.0 L). The filtrate was evaporated in vacuo to give a solid that was triturated with 10% MTBE in heptane (1.0 L) to give 40 (179.3 g, 84%) as a yellow solid.

### Example 10

### Synthesis of Di-Deutero Intermediate 43

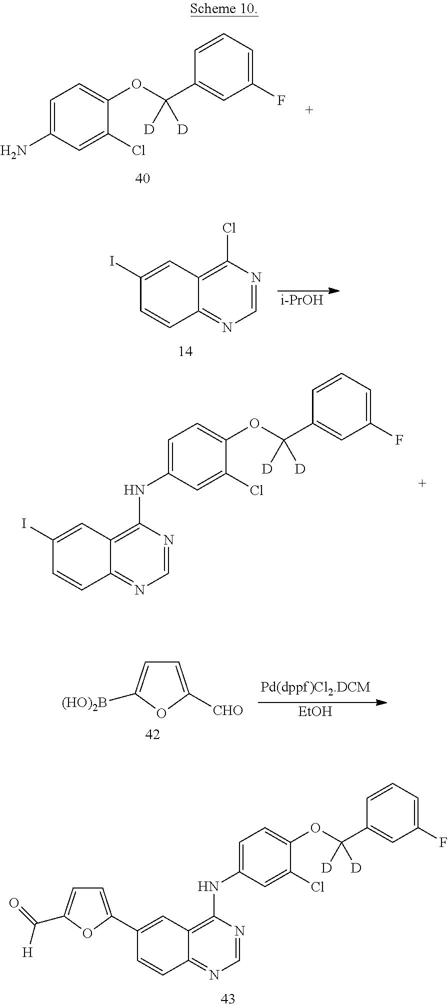

Scheme 10 depicts the synthesis of a common intermediate used to produce compounds of formula I, wherein Y^{4a} and Y^{4b} are simultaneously deuterium and Y^{3a} is hydrogen. The details of Scheme 10 are set forth below.

[3-Chloro-4-(3-fluoro-α,α-d₂-benzyloxy)phenyl](6-iodoquinazolin-4-yl)amine hydrochloride (41). To a suspension of 14 (ca. 400 mmol) in 2-propanol (2.2 L) was added 40 (104.5 g, 412.0 mmol). The reaction mixture was stirred under reflux conditions for 4 h, cooled to room temperature, and stirred overnight. The resulting precipitate was removed by filtration, washed with acetone (1.2 L), and dried at 60° C. for 2 h to give 41 (191 g, 88%) as a yellow solid.

5-{4-[3-Chloro-4-(3-fluoro-α,α-d₂-benzyloxy)phenylamino]quinazolin-6-yl}furan-2-carbaldehyde (43). To a suspension of 41 (24.2 g, 47.7 mmol) in ethanol (360 mL, purged with nitrogen prior to use) was added triethylamine (26.8 mL, 190.8 mmol, purged with nitrogen prior to use) followed by 5-formylfuran-2-boronic acid (42; 10.0 g, 71.6 mmol) and Pd(dppf)Cl₂.DCM (1.57 g). The reaction mixture was stirred under reflux conditions for 2 h and the volatiles were removed under reduced pressure. The crude residue was triturated with water (500 mL) and then with methanol (500 mL) and dried at 60° C. to give 43 (18.0) as a tan solid.

### Example 11

### Synthesis of Boronic Acid Reagent 46

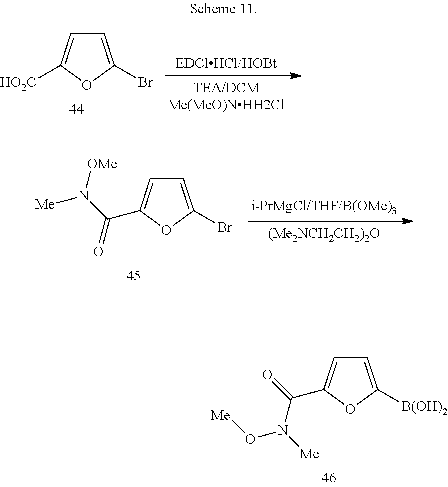

Scheme 11 depicts the synthesis of a boronic acid reagent used in the further synthesis of compounds of this invention. The details of the depicted scheme are set forth below.

5-Bromo-furan-2-carboxylic acid methoxy-methyl-amide (45) To a suspension of EDCI.HCl (75.0 g, 391.6 mmol) in DCM (800 mL) in an ice/water bath was added triethylamine (124.8 mL, 890.0 mmol). 5 minutes later 5-bromo-2-furoic acid (44; 68 g, 356.0 mmol) and anhydrous HOBt (52.9 g, 391.6 mmol) were added. The reaction mixture was stirred another 10 min in the ice/water bath and O-methyl-N-methylhydroxylamine hydrochloride (38.2 g, 391.6 mmol) was added. The reaction was allowed to warm to room temperature automatically overnight. The reaction was quenched with water (1.5 L) and the layers were split. The aqueous layer was extracted with DCM (2×500 mL) and the combined organic layers were dried over sodium sulfate and evaporated in vacuo to give a crude oil that was purified on a Silica-gel column with 1:4 EtOAc/heptane as eluent to give 45 (78 g, 85%) as a pale yellow oil.

5-(methoxy(methyl)carbamoyl)furan-2-ylboronic acid (46) To a solution of bis[2-(N,N-dimethylamino)ethyl]ether (76.8 g, 480 mmol) in anhydrous THF (2.0 L) at 15° C. was added 2.0 M isopropylmagnesium chloride (240 mL, 480 mmol) in THF over a period of 15 min. The mixture was stirred for 10 min followed by addition of 45 (93.6 g, 400 mmol) in THF (100 mL), keeping the internal temperature below 15° C. The resulting mixture was stirred for 20 min at ambient temperature, trimethyl borate (83.2 g, 800 mmol) was added at 0° C. and stifling was continued at 0° C. for 30 min. The reaction was quenched with 1.0 M hydrochloric acid, the mixture was brought to pH 6, and then saturated with sodium chloride and extracted with EtOAc (3×1.0 L). The combined organic layers were dried over anhydrous sodium sulfate and evaporated in vacuo. The crude solid was triturated with 1:1 EtOAc/heptane (1.0 L) and dried under vacuum at 60° C. to give 46 (33.3 g) as a yellow solid.

### Example 12

### Synthesis of Tri-Deuterated Intermediate 48

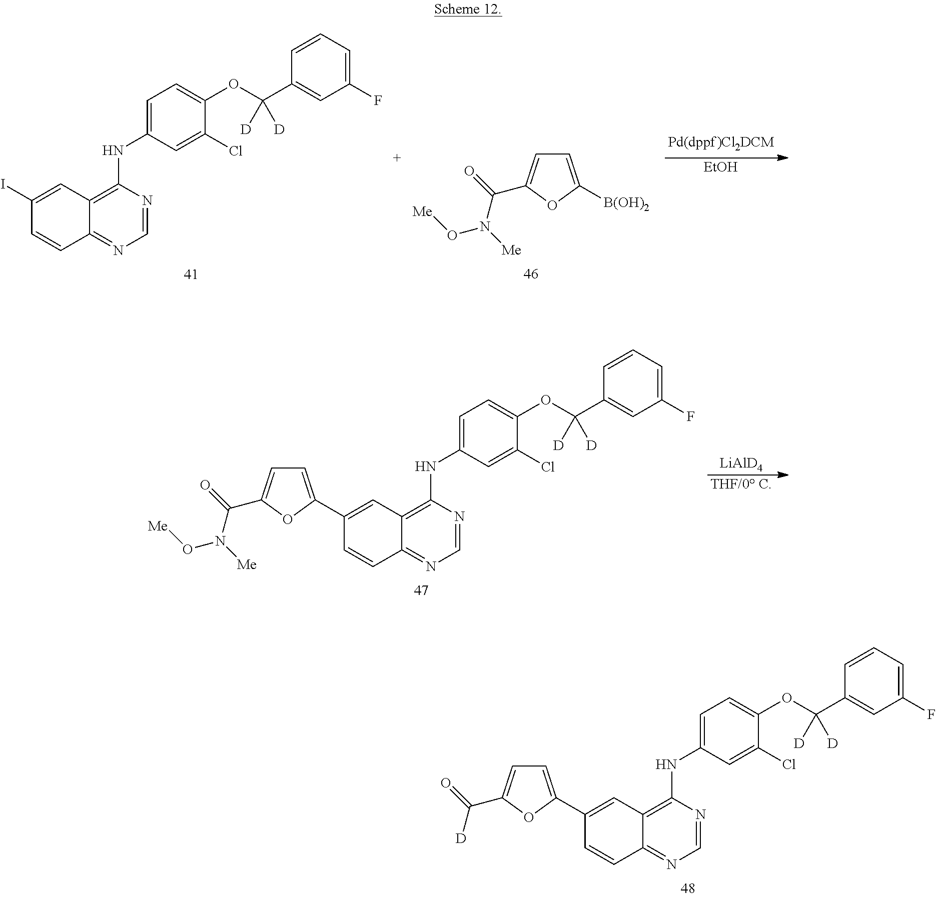

Scheme 12 depicts the synthesis of a trideuterated intermediate used in the synthesis of compounds of this invention wherein Y^{4a}, Y^{4b} and Y^{3a} are simultaneously deuterium. The details of scheme 12 are set forth below.

5-{4-[3-Chloro-4-(3-fluoro-α,α-d₂-benzyloxy)-phenylamino]-quinazolin-6-yl}-furan-2-carboxylic acid methoxy-methyl-amide (47) To a suspension of 41 (42.5 g, 83.6 mmol) in ethanol (630 mL, purged with nitrogen prior to use) was added triethylamine (43.9 mL, purged with nitrogen prior to use) followed by 46 (33.3 g, 167.2 mmol) and Pd(dppf)Cl₂.DCM (2.75 g). The reaction mixture was stirred under reflux conditions for 2 h and the volatiles were removed under reduced pressure. The crude residue was purified on a silica gel column with 1:1 EtOAc/heptane as eluent to give 47 (9.7 g) as a yellow solid.

5-{4-[3-Chloro-4-(3-fluoro-α,α-d₂-benzyloxy)phenylamino]quinazolin-6-yl}furan-2-carbaldehyde-d (48) To a solution of 47 (9.8 g, 18.3 mmol) in THF (720 mL), cooled in an ice/water/salt bath, was added lithium aluminum deuteride (768 mg, 18.3 mmol) portionwise allowing the internal temperature to remain below 5° C. The reaction was stirred for 1 h in the cooling bath, and then was quenched sequentially with deuterium oxide (1 mL), 15 wt % NaOD in deuterium oxide (1 mL), and deuterium oxide (1 mL). The resulting mixture was filtered over celite, and the filter cake washed with THF (300 mL). The filtrate was reduced in vacuo to give 48 as a yellow solid in quantitative yield.

### Example 13

### Synthesis of Compound 104 Tosylate Salt

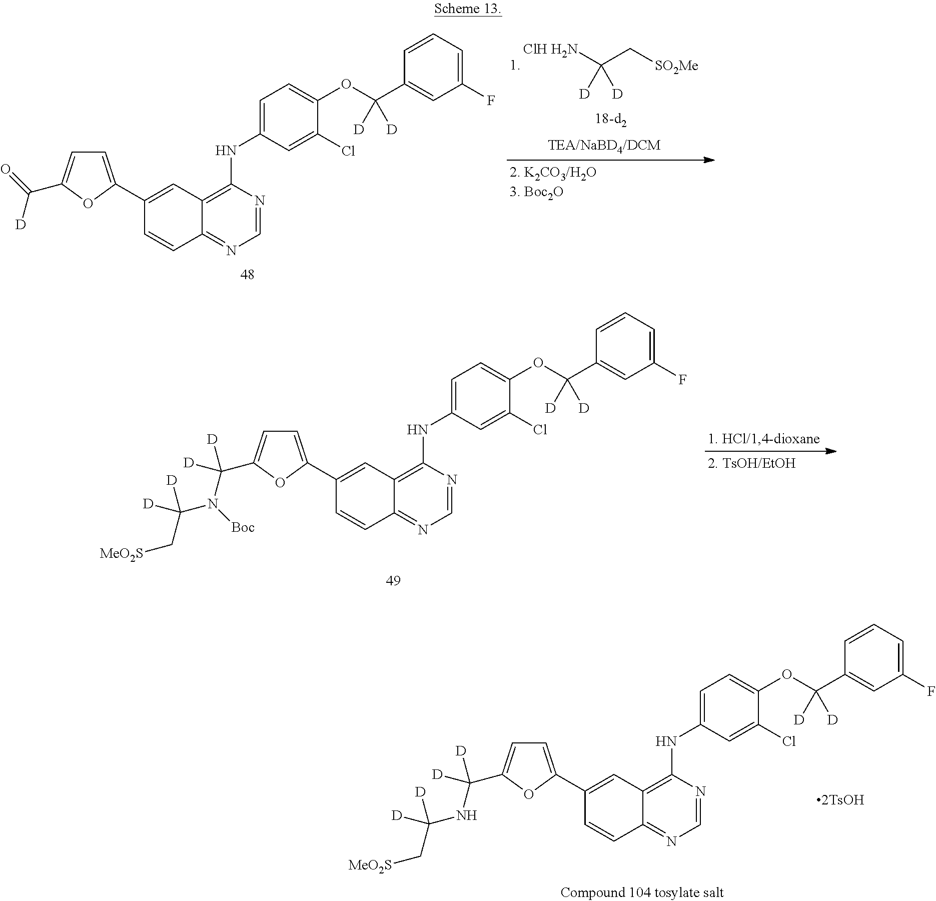

Scheme 13 depicts the preparation of Compound 104 tosylate salt. The details of Scheme 13 are set forth below.

Intermediate 49. To a suspension of amine hydrochloride 18-d2 (14.0 mmol) in DCM (300 mL) was added triethylamine (3.5 mL, 25.2 mmol). The suspension was stirred for 10 min after which time 48 (8.4 mmol) and sodium sulfate (20 g) were added. The reaction mixture was stirred for 3 h at room temperature, and then sodium borodeuteride (1.17 g, 28.06 mmol) was added portionwise. The resulting mixture was stirred overnight at room temperature and quenched with 10 wt % potassium carbonate in deuterium oxide (300 mL) at 0° C. After 20 min Boc₂O (14.0 mmol) was added and the reaction was stirred at room temperature for 2 h. The layers were separated and the aqueous layer was extracted with ethyl acetate (2×300 mL). The combined organic layers were dried over anhydrous sodium sulfate and concentrated in vacuo to afford a crude residue that was purified on a silica-gel column with ethyl acetate as eluent to afford 49 as a viscous oil or foam.

Compound 104 Tosylate Salt. To a solution of 49 in 1,4-dioxane (3 mL per mmol of 49) at room temperature was added 4.0 M HCl in 1,4-dioxane (10 mL per mmol of 49). The reaction mixture was stirred for 3 h at room temperature and concentrated in vacuo. The resulting yellow solid was suspended in ethyl acetate (300 mL) and neutralized with 10 wt % potassium carbonate in water (100 mL). The layers were separated and the aqueous layer was extracted with ethyl acetate (2×). The combined organic layers were dried over anhydrous sodium sulfate and concentrated in vacuo to yield a tan foam. The foam was dissolved in THF (4 mL per mmol of 49) and added to a solution of p-toluenesulfonate monohydrate (2.5 eq) in absolute ethanol (14 mL per mmol of 49) at 60° C. The suspension was stirred under reflux conditions for 1 h then was cooled to room temperature. The precipitate was collected by suction filtration, washed with a small amount of absolute ethanol, and then dried overnight at 60° C. to afford Compound 104 tosylate salt as a yellow solid. The average yield was ˜70% from the aldehyde.

¹H-NMR (300 MHz, DMSO-d₆): δ 2.25 (s, 6H), 3.11 (s, 3H), 3.53 (s, 2H), 6.87 (d, J=3.5, 1H), 7.07 (d, J=8.0, 4H), 7.16-7.34 (m, 5H), 7.45 (d, J=8.2, 4H), 7.46 (d, J=8.2, 1H), 7.59 (dd, J₁=8.9, J₂=2.5, 1H), 7.85 (d, J=2.3, 1H), 7.90 (d, J=8.8, 1H), 8.39 (dd, J₁=8.8, J₂=1.5, 1H), 8.91 (s, 1H), 9.00 (s, 1H), 9.25 (bs, 1H), 11.33 (bs, 1H). ¹³C-NMR (75 MHz, DMSO-d₆): δ 21.46, 41.37, 50.05, 110.08, 114.67, 114.72, 114.86, 115.40, 115.69, 115.82, 119.10, 121.91, 124.11, 125.24, 126.14, 126.99, 128.80, 130.21, 130.86, 131.29, 131.40, 138.53, 139.87, 139.96, 145.96, 147.25, 152.46, 153.08, 160.12, 161.25, 164.49. HPLC (method: 20 mm C18-RP column—gradient method 2-95% ACN in 4 min with 2 min hold at 95% ACN; Wavelength: 254 nm): retention time: 4.15 min. MS (M+H⁺): 587.1. Elemental Analysis (C₄₃H₃₆D₆ClFN₄O₁₀S₃.H₂0): Calculated: C=54.39, H=4.67, F=2.00, N=5.90. Found: C=54.19, H=4.32, F=2.02, N=5.76

### Example 14

### Synthesis of Compound 105 Tosylate Salt

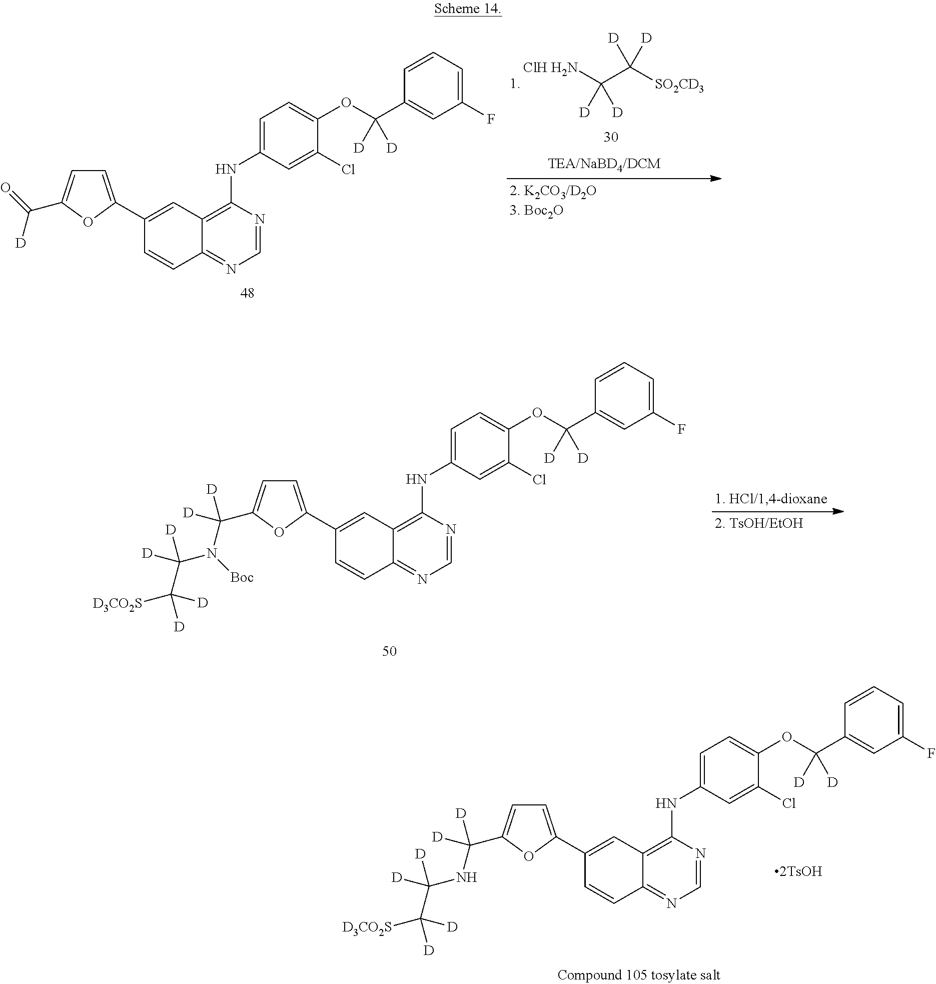

Scheme 14 depicts the preparation of Compound 105 tosylate salt. The details of Scheme 14 are set forth below.

Intermediate 50. Intermediate 50 is synthesized in an analagous manner to intermediate 49, except for the use of amine hydrochloride 30 in place of 18-d2.

Compound 105 Tosylate Salt. To a solution of 50 (1.0 eq) in 1,4-dioxane (3 mL per mmol of 50) at room temperature was added 4.0 M HCl in 1,4-dioxane (10 mL per mmol of 50). The reaction mixture was stirred 3 h at room temperature then concentrated in vacuo. The resulting yellow solid was suspended in ethyl acetate (40 mL per mmol of 50) and neutralized with 10 wt % potassium carbonate in deuterium oxide (100 mL). The layers were separated and the aqueous layer was extracted with ethyl acetate (2×). The combined organic layers were dried over anhydrous sodium sulfate and concentrated in vacuo to give a tan foam. The foam was dissolved in THF (4 mL per mmol of 50) then added to a solution of p-toluenesulfonate monohydrate (2.5 eq) in absolute ethanol (14 mL per mmol of 50) at 60° C. The suspension was stirred under reflux conditions for 1 h then cooled to room temperature. The precipitate was collected by suction filtration, washed with a small amount of absolute ethanol, and then dried overnight at 60° C. to afford Compound 105 tosylate salt as a yellow solid. The yield was ˜70% from the aldehyde.

¹H-NMR (300 MHz, DMSO-d₆): δ 2.25 (s, 6H), 6.87 (d, J=3.5, 1H), 7.08 (d, J=8.5, 4H), 7.15-7.33 (m, 5H), 7.45 (d, J=8.2, 4H), 7.46 (d, J=8.2, 1H), 7.58 (dd, J₁=9.1, J₂=2.6, 1H), 7.84 (d, J=2.6, 1H), 7.90 (d, J=8.8, 1H), 8.40 (dd, J₁=8.8, J₂=1.5, 1H), 8.92 (s, 1H), 9.01 (s, 1H), 9.28 (bs, 1H), 11.38 (bs, 1H). ¹³C-NMR (75 MHz, DMSO-d₆): δ 21.45, 110.13, 114.67, 114.73, 114.85, 114.96, 115.41, 115.69, 115.83, 119.12, 121.92, 124.14, 125.31, 126.14, 127.06, 128.81, 130.30, 130.78, 131.29, 131.40, 138.55, 139.87, 139.97, 145.92, 147.28, 152.54, 153.04, 160.19, 161.26, 164.50. HPLC (method: 20 mm C18-RP column—gradient method 2-95% ACN in 4 min with 2 min hold at 95% ACN; Wavelength: 254 nm): retention time: 4.23 min. MS (M+H⁺): 592.2.

### Example 15

### Synthesis of Compound 106 Tosylate Salt

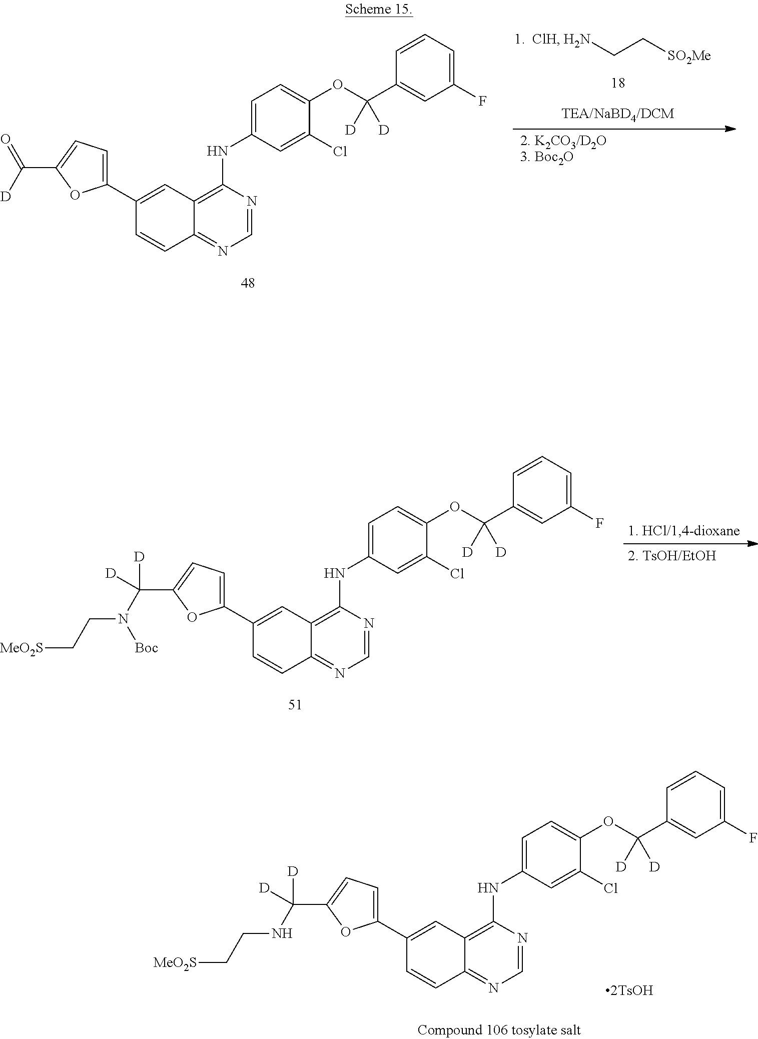

Scheme 15 depicts the preparation of Compound 106 tosylate salt. The details of Scheme 15 are set forth below.

Intermediate 51. Intermediate 51 was synthesized in an analagous manner to intermediate 49, except for the use of amine hydrochloride 18 in place of 18-d2.

Compound 106 Tosylate Salt. Compound 106 tosylate salt was produced in an analagous manner to Compound 104 tosylate salt, except for the use of intermediate 51 in place of 49.

¹H-NMR (300 MHz, DMSO-d₆): δ 2.25 (s, 6H), 3.11 (s, 3H), 3.52-3.57 (m, 4H, partially obscured by H₂O peak), 6.87 (d, J=3.6, 1H), 7.07 (d, J=8.5, 4H), 7.18-7.34 (m, 5H), 7.45 (d, J=8.2, 4H), 7.46 (d, J=7.9, 1H), 7.59 (dd, J₁=8.8, J₂=2.5, 1H), 7.84 (d, J=2.3, 1H), 7.90 (d, J=8.8, 1H), 8.39 (dd, J₁=8.8, J₂=1.5, 1H), 8.91 (s, 1H), 9.00 (s, 1H), 9.26 (bs, 1H), 11.36 (bs, 1H). ¹³C-NMR (75 MHz, DMSO-d₆): δ 21.46, 41.35, 50.20, 110.05, 114.67, 114.78, 114.86, 114.96, 115.69, 115.84, 119.10, 121.91, 124.14, 125.24, 126.14, 127.00, 128.80, 130.22, 130.87, 131.29, 131.40, 138.54, 139.97, 145.95, 147.25, 152.46, 153.09, 160.12, 161.26, 164.50. HPLC (method: 20 mm C18-RP column—gradient method 2-95% ACN in 4 min with 2 min hold at 95% ACN; Wavelength: 254 nm): retention time: 4.24 min. MS (M+H⁺): 585.0. Elemental Analysis (C₄₃H₃₈D₄ClFN₄O₁₀S₃): Calculated: C=55.57, H=4.55, F=2.04, N=6.03. Found: C=55.46, H=4.30, F=2.07, N=5.94.

### Example 16

### Synthesis of Compound 107 Tosylate Salt

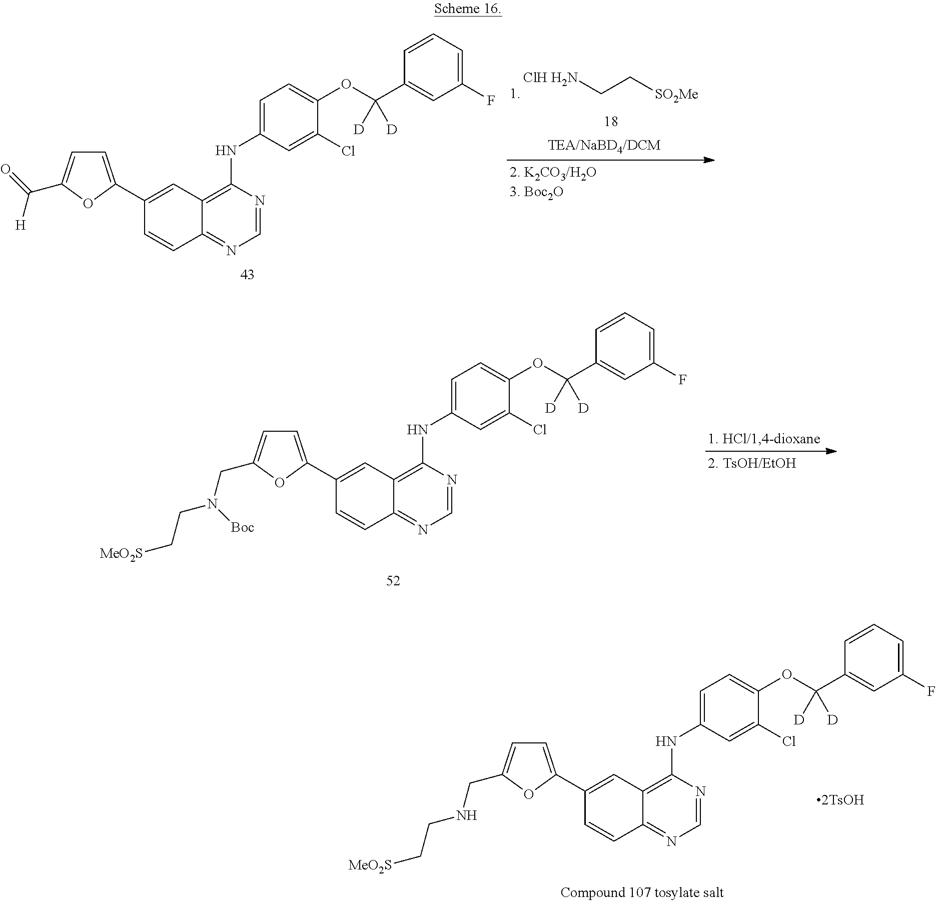

Scheme 16 depicts the preparation of Compound 107 tosylate salt. The details of Scheme 16 are set forth below.

Intermediate 52. To a suspension of the amine hydrochloride 18 (25 mmol) in DCM (500 mL) was added triethylamine (4.7 mL, 31.8 mmol). The mixture was stirred for 1 h at which time 43 (7.1 g, 15.0 mmol) was added and stifling was continued for 1 h at room temperature. Sodium triacetoxyborohydride (9.7 g, 40.1 mmol) was added portionwise and the resulting mixture was stirred overnight at room temperature, quenched with 10 wt % potassium carbonate in water (300 mL) at 0° C., and then stirred for 20 min. To the resulting mixture was added Boc₂O (25 mmol) with stirring at room temperature for 2 h. The layers were separated and the aqueous layer was extracted with ethyl acetate (2×300 mL). The combined organic layers were dried over anhydrous sodium sulfate and concentrated in vacuo to afford a crude residue that was purified on a silica-gel column with ethyl acetate as eluent to afford 52 as a foam.

Compound 107 Tosylate Salt. Compound 107 tosylate salt wasproduced in an analagous manner to Compound 104 tosylate salt, except for the use of intermediate 52 in place of 49.

¹H-NMR (300 MHz, DMSO-d₆): δ 2.28 (s, 6H), 3.15 (s, 3H), 3.43-3.55 (m, 4H, partially obscured by H₂O peak), 4.46 (s, 2H), 6.90 (d, J=3.2, 1H), 7.11 (d, J=7.9, 4H), 7.18-7.24 (m, 2H), 7.34-7.37 (m, 3H), 7.47 (d, J=8.2, 4H), 7.52 (d, J=8.2, 1H), 7.64 (d, J=9.4, 1H), 7.91-7.94 (m, 2H), 8.38 (d, J=8.8, 1H), 8.89 (s, 1H), 8.99 (s, 1H), 9.29 (bs, 1H), 11.14 (bs, 1H). ¹³C-NMR (75 MHz, DMSO-d₆): δ 21.45, 41.37, 43.35, 50.21, 109.97, 114.68, 114.89, 115.82, 119.07, 121.91, 124.14, 125.10, 126.14, 126.87, 128.77, 130.07, 131.29, 131.40, 138.44, 139.88, 146.12, 147.26, 152.34, 153.16, 160.00, 161.26, 164.50. HPLC (method: 20 mm C18-RP column—gradient method 2-95% ACN in 4 min with 2 min hold at 95% ACN; Wavelength: 254 nm): retention time: 4.24 min. MS (M+H⁺): 583.2. Elemental Analysis (C₄₃H₄₀D₂ClFN₄O₁₀S₃.0.1H₂O): Calculated: C=55.58, H=4.58, Cl=3.82, F=2.04, N=6.03. Found: C=55.25, H=4.42, Cl=3.91, F=2.02, N=5.95.

### Example 17

### Synthesis of Compound 108 Tosylate Salt

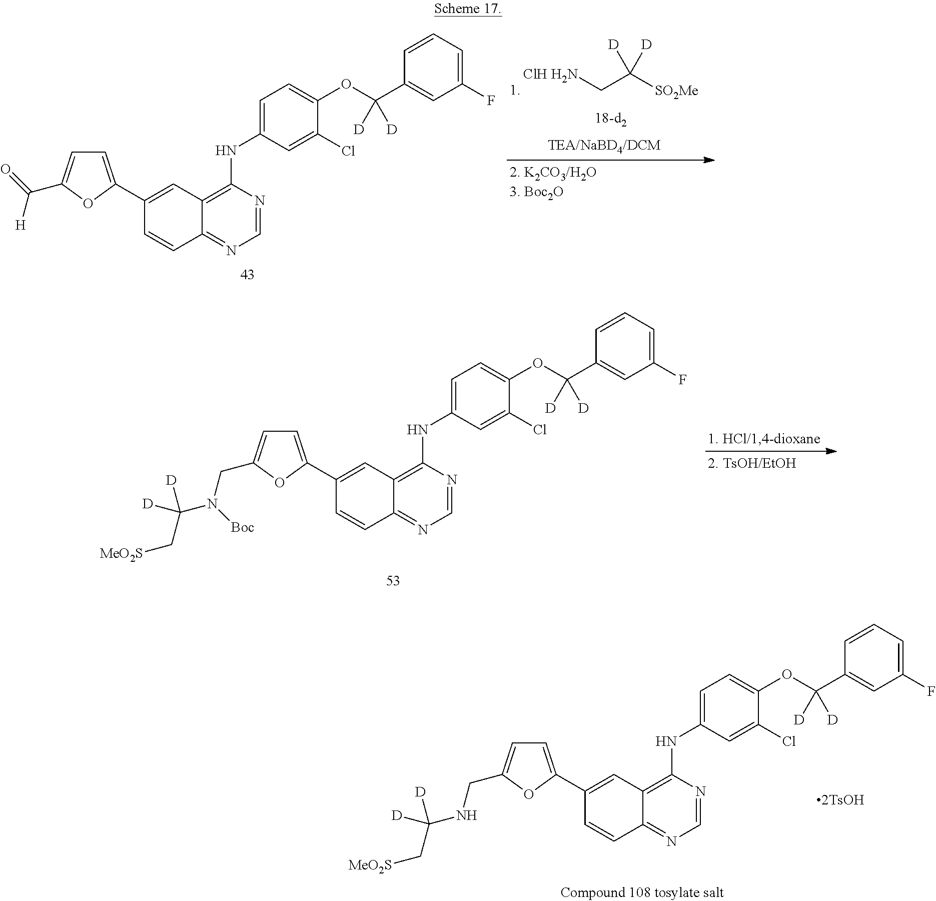

Scheme 17 depicts the preparation of Compound 108 tosylate salt. The details of Scheme 17 are set forth below.

Intermediate 53. Intermediate 53 was synthesized in an analagous manner to intermediate 52, except for the use of amine hydrochloride 18-d2 in place of 18.

Compound 108 Tosylate Salt. Compound 108 tosylate salt was produced in an analagous manner to Compound 104 tosylate salt, except for the use of intermediate 53 in place of 49.

¹H-NMR (300 MHz, DMSO-d₆): δ 2.29 (s, 6H), 3.14 (s, 3H), 3.56 (s, 2H, partially obscured by H₂O peak), 4.46, (s, 2H), 6.89 (s, 1H), 7.10 (d, J=7.0, 4H), 7.17-7.24 (m, 2H), 7.31-7.37 (m, 3H), 7.47 (d, J=8.2, 4H), 7.49 (d, J=8.2, 1H), 7.64 (d, J=9.1, 1H), 7.90-7.94 (m, 2H), 8.38 (d, J=8.5, 1H), 8.88 (s, 1H), 8.99 (s, 1H), 9.30 (bs), 11.15 (bs). ¹³C-NMR (75 MHz, DMSO-d₆): δ 21.46, 41.37, 50.05, 110.09, 114.67, 114.77, 114.86, 114.97, 115.40, 115.69, 115.82, 119.10, 121.91, 124.11, 125.26, 126.14, 127.01, 128.80, 130.23, 130.85, 131.29, 131.40, 131.93, 138.52, 139.87, 139.97, 145.99, 147.32, 152.06, 152.48, 153.07, 160.14, 161.26, 164.49. HPLC (method: 20 mm C18-RP column—gradient method 2-95% ACN in 4 min with 2 min hold at 95% ACN; Wavelength: 254 nm): retention time: 4.24 min. MS (M+H⁺): 585.0. Elemental Analysis (C₄₃H₃₈D₄ClFN₄O₁₀S₃.1.5H₂O): Calculated: C=54.00, H=4.74, Cl=3.71, F=1.99, N=5.86. Found: C=53.71, H=4.41, Cl=3.89, F=1.82, N=5.74.

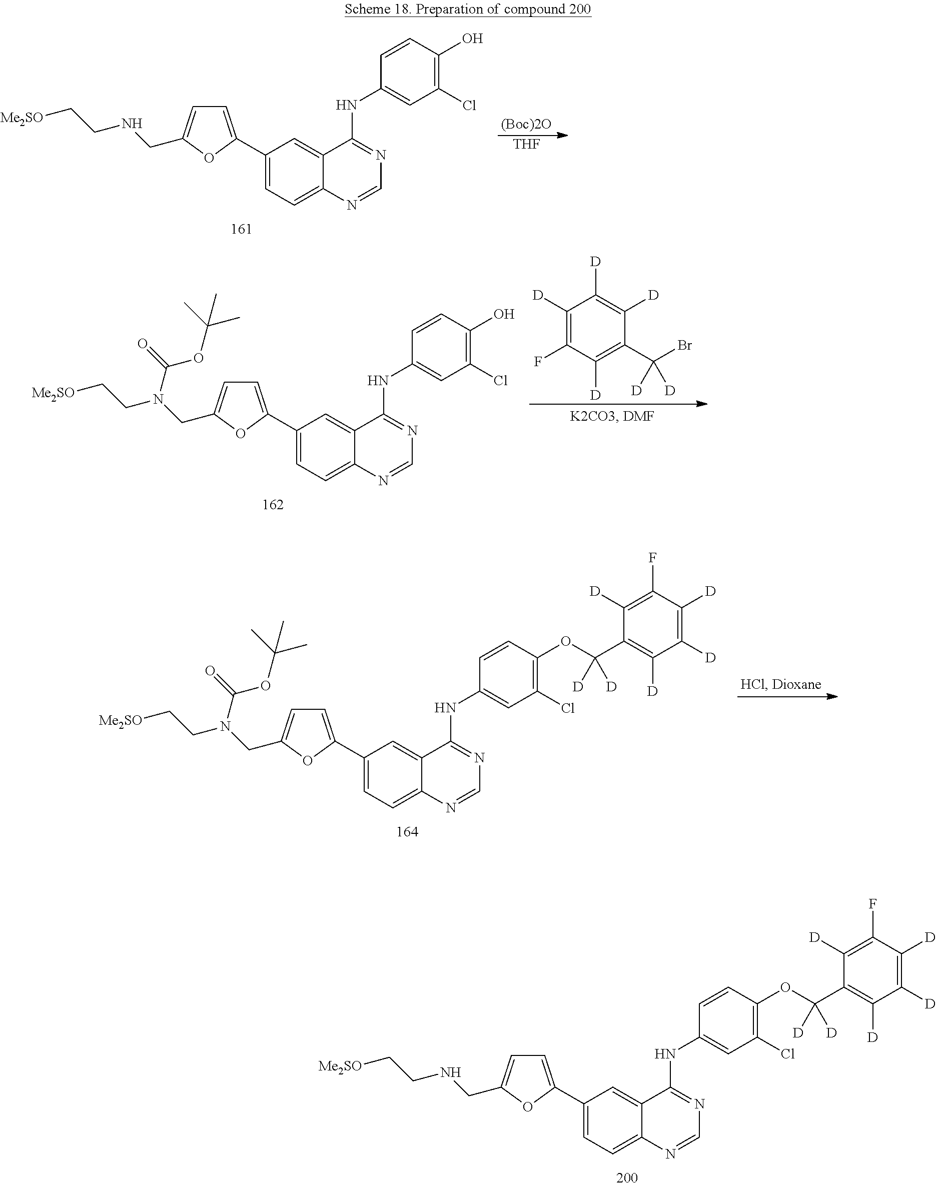

The details of each step in Scheme 18 are set forth below:

tert-butyl((5-(4-((3-chloro-4-hydroxyphenyl)amino)quinazolin-6-yl)furan-2-yl)methyl)(2-(methylsulfonyl)ethyl)carbamate (162): Di-tert-butyl dicarbonate (79 mg, 0.362 mmol) was added to a 100 mL round-bottom flask containing a tetrahydrofuran (5 mL) solution of phenol 161 (0.241 mmol), prepared as disclosed below. The reaction proceeded at ambient temperature until deemed complete by LCMS analysis. Volatiles were removed by concentration and the reaction was re-constituted in ethyl acetate. The organic layer was washed successively with saturated aqueous solutions of ammonium chloride and brine. After separation of phases the organic layers were dried over sodium sulfate, filtered, and concentrated to give a yellow-brown oil. Purification of the oil on silica gel eluting with methanol-dichloromethane (100% CH₂Cl₂ −5% CH₃OH:95% CH₂Cl₂) provided carbamate 162 as a brown oil (80 mg, 58% yield). MS(ESI) 573.1 [(M+H)⁺].

tert-butyl((5-(4-((3-chloro-4-((3-fluoro-2,4,5,6-tetradeuterophenyl)dideuteromethoxy)phenyl)amino)-quinazolin-6-yl)furan-2-yl)methyl)(2-(methylsulfonyl)ethyl)carbamate (164): A solution of carbamate 162 (25 mg, 0.044 mmol) in DMF (2 mL) was prepared in a 25 mL round bottom flask. Potassium carbonate (18 mg, 0.132 mmol) was then added as a single portion followed by a solution of 3-fluorobenzyl bromide-d6 (163), prepared as disclosed in Scheme 19 below, in DCM (1 mL, 0.06M). The reaction was heated with an oil bath to 45° C. until the reaction was deemed complete by LCMS. At this point the solvents were removed by concentration and the reaction mixture dried under reduced pressure to yield a brown oil. Purification of the oil on silica gel eluting with methanol-dichloromethane (100% CH₂Cl₂ −20% CH₃OH:80% CH₂Cl₂) provided ether 164 as a yellow oil (10 mg, 0.014 mmol, 33% yield). MS(ESI) 689.3 [(M+H)⁺].

Compound 200: Carbamate 164 (5.0 mg, 0.0073 mmol) was placed in a 5 mL round bottom flask followed by a solution of hydrogen chloride in 1,4-dioxane (75 μL, 4M). After 90 minutes, LCMS analysis indicated complete conversion to desired product. The recation was then concentrated and transferred to a tared 25 dram vial with a solution of acetonitirile and water (1:1). This mixture was frozen and then lyophilized overnight to give a yellow oil (˜4 mg). Semi-preparative mass directed HPLC purification afforded Compound 200 as a yellow powder (3.0 mg, 0.0051mmol, 70% yield). MS(ESI) 587.3 [(M+H)⁺].

Phenol 161 may be prepared either using the general procedure described in Scheme B hereinabove to obtain a compound of formula XVII or as follows:

Lapatinib (145 mg, 0.250 mmol.) was dissolved in an equal mixture of toluene and triflouroacetic acid (50 mL) in a 100 mL round-bottom flask. The solution was stirred at 80° C. for 8 h at which time LCMS analysis indicated completion. The solution was then cooled to ambient temperature and concentrated under reduced pressure to afford a yellow powder which contained desired phenol 1 as the major component as indicated by LCMS. This material was carried forward directly into the next reaction without further purification. MS(ESI) 473.1 [(M+H)⁺]

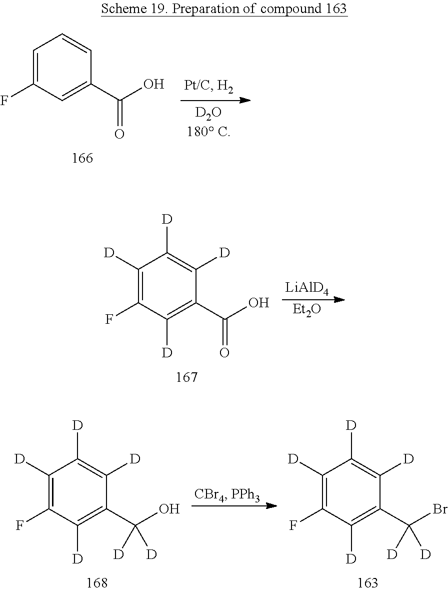

3-Fluorobenzoic acid-d4 (167): To a heavy-walled pressure flask containing a suspension of 3-fluorobenzoic acid (166) (500 mg, 3.57 mmol) in D₂O (30 mL, Cambridge Isotope Laboratories, 99.9 atom % D) was added 5 wt % platinum on activated carbon (100 mg). The reaction was flushed several times with nitrogen followed by hydrogen gas. The reaction was then allowed to stir under an atmosphere of hydrogen for 20 minutes. The vessel was then purged repeatedly with nitrogen then sealed, heated to 180° C. and stirred for 15 hours. The reaction was then cooled to room temperature and an aliquot was removed for LCMS analysis which indicated incomplete H/D exchange. Another portion of 5 wt % platinum on activated carbon (100 mg) was added and the reaction was again flushed with nitrogen several times followed by hydrogen gas. The reaction was then allowed to stir under an atmosphere of hydrogen for 20 minutes then purged repeatedly with nitrogen, sealed and heated to 180° C. and stirred for an additional 15 hours. The reaction was then cooled to room temperature and an aliquot was removed for LCMS analysis which indicated complete H/D exchange. 1M Sodium hydroxide (10 mL) was then added and the reaction was filtered through Celite® rinsing with excess 1M sodium hydroxide. The resulting aqueous filtrate was then acidified to pH<2 with 1M HCl and extracted with ethyl acetate (3×100 mL). The organic layers were then combined, dried with Na₂SO₄, filtered and concentrated under reduced pressure to afford 3-fluorobenzoic acid-d4 (167) (373 mg, 73% yield) as a white solid. MS (ESI) 143.1 [(M−H)⁻].

3-Fluorobenzyl alcohol-d6 (168): To a solution of 3-fluorobenzoic acid-d4 (167) (373 mg, 2.59 mmol) in diethylether (13 mL) at 0° C. was added lithium aluminum deuteride (131 mg, 3.11 mmol, Cambridge Isotope Laboratories, 99 atom % D) portionwise. The reaction was then stirred at room temperature for 3 hours then cooled to 0° C. and quenched by the sequential addition of water (131 mL), 15% sodium hydroxide (131 mL) then water (393 mL). The resulting white slurry was stirred at room temperature for 30 minutes then filtered through Celite® rinsing with excess diethylether. The filtrate was then concentrated under reduced pressure. The resuting clear oil (168, 342 mg, 100% yield) was used without further purification. MS (ESI) 115.2 [(M−OH)⁺].

3-Fluorobenzyl bromide-d6 (163): To a solution of 3-fluorobenzyl alcohol-d6 (168) (240 mg, 1.81 mmol) and carbon tetrabromide (660 mg, 1.99 mmol) in dichloromethane at 0° C. was added triphenylphosphine (475 mg, 1.81 mmol). The reaction was stirred at room temperature for 15 hours then diluted with diethylether, filtered through a plug of silica gel rinsing with excess diethylether, then concentrated under reduced pressure to afford 3-fluorobenzyl bromide-d6 (163, 1.81 mmol) which was used without further purification.

As is readily understood by one skilled in the art, the above procedure may be suitably modified to obtain other deuterated intermediates. For example, addition of lithium aluminum hydride to 167, followed by treatment of the resulting tetradeuterated benzyl alcohol with carbon tetrabromide and triphenylphosphine (analogously to the manner described hereinabove for 168) provides the corresponding tetradeuterated 3-fluorobenzyl bromide 169:

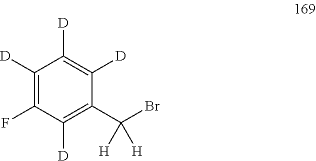

may be then used for the preparation of the compounds of the invention in accordance with Scheme B herein.

### Example 18

### Evaluation of Metabolic Stability in Human Liver Microsomes

The metabolic stability of the present compounds may be evaluated in one or more microsomal assays that are known in the art. See, for example, Obach, R. S. Drug Metab Disp 1999, 27, p. 1350 “Prediction of human clearance of twenty-nine drugs from hepatic microsomal intrinsic clearance data: An examination of in vitro half-life approach and nonspecific binding to microsomes”; Houston, J. B. et al., Drug Metab Rev 1997, 29, p. 891 “Prediction of hepatic clearance from microsomes, hepatocytes, and liver slices”; Houston, J. B. Biochem Pharmacol 1994, 47, p. 1469 “Utility of in vitro drug metabolism data in predicting in vivo metabolic clearance”; Iwatsubo, T et al., Pharmacol Ther 1997, 73, p. 147 “Prediction of in vivo drug metabolism in the human liver from in vitro metabolism data”; and Lave, T. et al., Pharm Res 1997, 14, p. 152 “The use of human hepatocytes to select compounds based on their expected hepatic extraction ratios in humans”; each of which are incorporated herein in their entirety.

The objectives of this study were to determine the metabolic stability of the test compounds in pooled liver microsomal incubations and to perform full scan LC-MS analysis for the detection of major metabolites. Samples of the test compounds, exposed to pooled human liver microsomes, were analyzed using HPLC-MS (or MS/MS) detection. For determining metabolic stability, multiple reaction monitoring (MRM) was used to measure the disappearance of the test compounds. For metabolite detection, Q1 full scans were used as survey scans to detect the major metabolites.

Experimental Procedures: Human liver microsomes were obtained from Absorption Systems L.P. (Exton, Pa.). Details about the matrices used in the experiments are shown below. The incubation mixture was prepared as follows:

Reaction Mixture Compositions:

| | | |
|---|---|---|
| Liver Microsomes | 0.1-2 | mg/mL |
| NADPH | 1 | mM |
| Potassium Phosphate, pH 7.4 | 100 | mM |
| Magnesium Chloride | 10 | mM |
| Test Compound (Tosylate Salts | 0.1-1.0 | μM |
| of Lapatinib, and Compounds | | |
| 100-108) | | |

For each individual experiment, the same amount of compound and the same amount of microsomes were used. Amounts of each varied from experiment to experiment.

Incubation of Test Compounds with Liver Microsomes: The reaction mixture, minus cofactors, was prepared. An aliquot of the reaction mixture (without cofactors) was incubated in a shaking water bath at 37° C. for 3 minutes. Another aliquot of the reaction mixture was prepared as the negative control. The test compound was added into both the reaction mixture and the negative control at a final concentration of 0.1-1 μM, depending upon the experiment. An aliquot of the reaction mixture was prepared as a blank control, by the addition of plain organic solvent (not the test compound). The reaction was initiated by the addition of cofactors (not into the negative controls), and then incubated in a shaking water bath at 37° C. Aliquots (200 μL) were withdrawn in triplicate at 0, 10, 20, 40, and 60 minutes and combined with 800 μL of ice-cold 50/50 acetonitrile/dH₂O to terminate the reaction. The positive controls, testosterone and propranolol, were run simultaneously with the test compounds in separate reactions.

All samples were analyzed using LC-MS (or MS/MS). An LC-MRM-MS/MS method was used for metabolic stability. Also, Q1 full scan LC-MS methods were performed on the blank matrix and the test compound incubation samples. The Q1 scans served as survey scans to identify any sample unique peaks that might represent the possible metabolites. The masses of these potential metabolites can be determined from the Q1 scans.

A similar experiment was performed with rat liver microsomes using the tosylate salts of lapatinib, Compound 101 and Compound 102.

The results of these microsomal assays (data not shown) showed no significant differences between the stability of the two deuterated compounds as compared to lapatinib. Without being bound by theory, the inventors believe that these microsomal stability experiments were confounded by the low solubility and high-protein binding properties of lapatinib and the compounds of this invention.

### Example 19

### Evaluation of Metabolic Stability in CYP3A4 SUPERSOMES™

Because the SUPERSOME™ system does not require as high a concentration of compound it was selected as an alternative to study the comparative stability of the compounds of this invention and lapatinib. This lower protein concentration avoids the non-specific binding of lapatinib and the test compounds of this invention to other microsomal proteins.

Human CYP3A4+P450 Reductase SUPERSOMES™ were purchased from GenTest (Woburn, Mass., USA). A 1.0 mL reaction mixture containing 25 pmole of SUPERSOMES™, 2.0 mM NADPH, 3.0 mM MgCl, and 0.1 μM of various compounds of Formula I (the tosylate salt of each of compounds 101, 102, 104, 105, 106, 107 or 108) in 100 mM potassium phosphate buffer (pH 7.4) was incubated at 37° C. in triplicate. Positive controls contained 0.1 μM lapatinib tosylate salt instead of a compound of formula I. Negative controls used Control Insect Cell Cytosol (insect cell microsomes that lacked any human metabolic enzyme) purchased from GenTest (Woburn, Mass., USA). Aliquots (50 μL) were removed from each sample and placed in wells of a multi-well plate at 0, 2, 5, 7, 12, 20, and 30 minutes and to each was added 50 L of ice cold acetonitrile with 3 μM haloperidol as an internal standard to stop the reaction.

Plates containing the removed aliquots were then placed in −20° C. freezer for 15 minutes to cool. After cooling, 100 μL of deionized water was added to all wells in the plate. Plates were then spun in the centrifuge for 10 minutes at 3000 rpm. A portion of the supernatant (100 μL) was then removed, placed in a new plate and analyzed using Mass Spectrometry.

The stability of each tested compound after 30 minutes is shown in Table 2, below.

TABLE 2
Stability of Compounds of Formula Ia
in CYP3A4 SUPEROMES ™.

| Compound | % remaining after 30 min |
|---|---|
| Lapatinib | 7.28 ± 0.361 |
| 101 | 9.32 ± 0.448 |
| 102 | 8.20 ± 0.486 |
| 104 | 10.1 ± 0.365 |
| 105 | 13.8 ± 0.493 |
| 106 | 13.1 ± 0.827 |
| 107 | 10.4 ± 0.270 |
| 108 | 11.3 ± 0.633 |

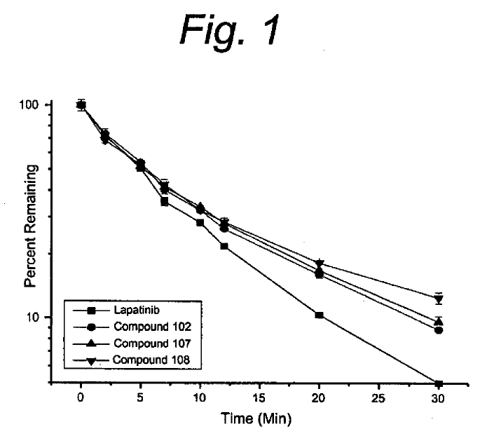

The stability of each tested compound after 30 minutes is shown in Table 2, below. FIG. 1 shows the time course of metabolism for each of the tosylate salts of lapatinib, Compound 102, Compound 107 and Compound 108 in this assay.

These results confirm that the deuterated compounds of the present invention are more resistant to cytochrome P450 oxidation than lapatinib and thus may have either an advantageously longer lasting effect when administered to human subjects and/or may be administered in lower dosages than lapatinib while providing the same therapeutic effect, thus avoiding undesirable side effects.

### Example 20

### Evaluation of Pharmacokinetics in Rats

Eighteen Sprague-Dawley rats were divided into three groups of 6 rats each to test and compare the pharmacokinetic fate of intravenous doses of the tosylate salts of lapatinib, Compound 101, Compound 102 and Compound 104.

Rats were anesthetized using pentobarbital (IP 40 mg/kg) prior to administration of compound. Separate 2 mg/mL solutions of the tosylate salts of lapatinib, Compound 101 and Compound 102 in 10% DMSO/90% H₂O were prepared. The rats were administered a single bolus 2 mg/kg dose of compound via jugular cannula, followed by a wash-out with saline. Blood samples (0.25 mL) were taken from the jugular at 5, 15 and 30 minutes and at 1, 2, 4, 6, 9, 12, and 24 hours post-dosing. Blood samples were centrifuged within 15 minutes of removal from the animal, centrifuged, and the plasma fraction removed and stored at −20° C. until analysis. Samples were analyzed by LC-MS.

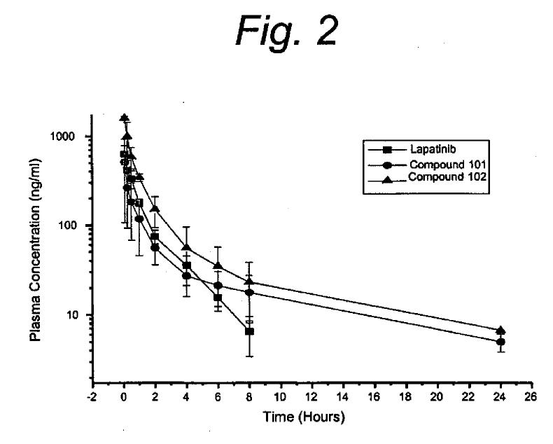

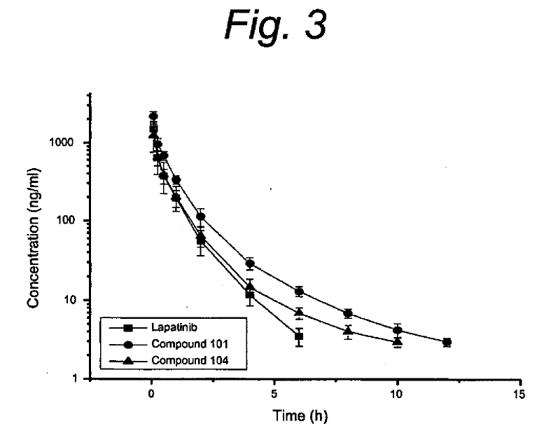

FIGS. 2 and 3 show the results of these experiments. Both of Compounds 101 and 102 demonstrated significantly longer half-lives than lapatinib (FIG. 2). Similarly Compound 104 also demonstrated a longer half-life than Lapatinib (FIG. 3). The calculated half life for lapatinib was 1.0±0.05 h. The half-lives for Compound 101 and 104 were 2.3±0.2 h and 2.3±0.3 h, respectively.

A similar experiment using oral dosing (20 mg/kg) of the tosylate salts of lapatinib, Compound 101 and Compound 102 (data not shown) demonstrated similar results with both Compound 101 and Compound 102 exhibiting longer half lives than lapatinib.

### Example 22

### In Vitro Biological Activity

The tosylate salts of Compounds 101, 102 and 103, as well as lapatinib were assayed for various kinase activities, as well as for their affect on cell proliferation. Assays were performed by Cerep (Redmond, Wash. USA) as described below.

The EGFR Kinase assay (Cerep catalog ref:768-E) was performed according to the methods set forth in Weber Wet al., J Biol Chem, 1984, 259:14631-36. EGFR kinase used in the assay was obtained from A-431 cells. Varying concentrations of test compound (0.1 nM, 1 nM, 3 nM, 10 nM, 30 nM, 100 nM, 300 nM, and 1 μM) were incubated with the kinase, ATP and 0.1 μM of the biotinylated peptide biotinyl-βAβAβAAEEEEYFELVAKKK at 22° C. for 30 minutes. Production of phospho-biotinyl-βAβAβAAEEEEYFELVAKKK was detected by Homogeneous Time Resolved Fluorescence (HTRF®).

The HER2 kinase assay (Cerep catalog ref:768-her2) was performed according to the methods set forth in Qian X et al., Proc Natl Acad Sci USA, 1992, 89:1330-34. Recombinant human HER2 kinase expressed in insect cells was used in this assay. Varying concentrations of test compound (0.1 nM, 1 nM, 3 nM, 10 nM, 30 nM, 100 nM, 300 nM, and 1 μM) were incubated with the kinase, ATP and 0.6 μM of the biotinylated peptide biotinyl-βAβAβAAEEEEYFELVAKKK at 22° C. for 30 minutes. Production of phospho-biotinyl-βAβAβAAEEEEYFELVAKKK was detected by Homogeneous Time Resolved Fluorescence (HTRF®).

The HER4 kinase assay (Cerep catalog ref:768-her4) was performed according to the methods set forth in Plowman G D et al., Proc Natl Acad Sci USA, 1993, 90:1746-50. Recombinant human HER2 kinase expressed in insect cells was used in this assay. Varying concentrations of test compound (0.1 nM, 1 nM, 3 nM, 10 nM, 30 nM, 100 nM, 300 nM, and 1 μM) were incubated with the kinase, ATP and 0.6 μM of the biotinylated peptide biotinyl-βAβAβAAEEEEYFELVAKKK at 22° C. for 30 minutes. Production of phospho-biotinyl-βAβAβAAEEEEYFELVAKKK was detected by HTRF®.

The cell proliferation assay (Cerep catalog ref:791-4) was performed according to the methods set forth in Handler J A et al., J Biol Chem, 1990, 265:3669-73. A-431 cells were stimulated with EGF (1 ng/ml) in the presence of [³H]-thymidine and various concentrations of test compound (0.1 nM, 1 nM, 3 nM, 10 nM, 30 nM, 100 nM, 300 nM, and 1 μM). After 24 hours of growth at 37° C., the cells were harvested and [³H]-thymidine incorporation measured by scintillation counting.

The results of these assay are shown in Table 3, below:

TABLE 3
Biological Activity of Compounds of Formula Ia

| | EGFR | HER2 | HER4 | Cell |
|---|---|---|---|---|
| | Kinase | Kinase | Kinase | Proliferation |
| Compound | (nM) | (μM) | (μM) | (nM) |
| Lapatinib | 230 | 4.0 | 2.3 | 670 |
| 101 | 120 | 3.2 | 2.3 | 610 |
| 102 | 260 | 2.7 | 1.9 | 180 |
| 103 | 170 | 5.0 | 3.1 | 330 |

These results demonstrate that the compounds of the present invention have similar potencies to lapatinib against the desired kinase targets, as well as on inhibiting cell proliferation.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of specific embodiments. It will be apparent to those of ordinary skill in the art that various modifications and equivalents can be made without departing from the spirit and scope of the invention.

What is claimed is:

1. A compound of Formula Ia: 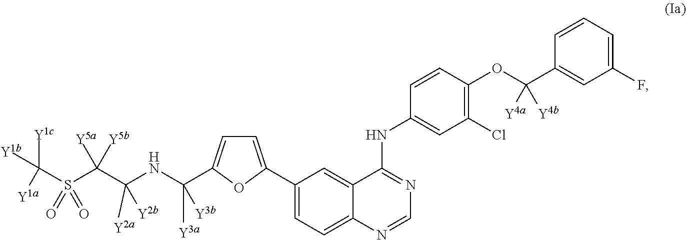
or a salt thereof;
wherein each Y is independently selected from hydrogen and deuterium; and at least one Y is deuterium.

2. The compound of claim 1, wherein each Y bound to a common carbon atom is the same.

3. The compound of claim 2, wherein Y^{4a} and Y^{4b} are simultaneously deuterium.

4. The compound of claim 3, selected from any one of the compounds set forth in the table below:

| Cmpd | Y^{1a} | Y^{1b} | Y^{1c} | Y^{2a} | Y^{2b} | Y^{3a} | Y^{3b} | Y^{4a} | Y^{4b} | Y^{5a} | Y^{5b} |
|---|---|---|---|---|---|---|---|---|---|---|---|
| 104 | H | H | H | D | D | D | D | D | D | H | H |
| 105 | D | D | D | D | D | D | D | D | D | D | D |
| 106 | H | H | H | H | H | D | D | D | D | H | H |
| 107 | H | H | H | H | H | H | H | D | D | H | H |
| 108 | H | H | H | D | D | H | H | D | D | H | H |

or a pharmaceutically acceptable salt of any one of the foregoing.

5. The compound of claim 4, wherein the compound is compound 105, 106, or 108, or a pharmaceutically acceptable salt of any one of the foregoing.

6. A compound of formula II 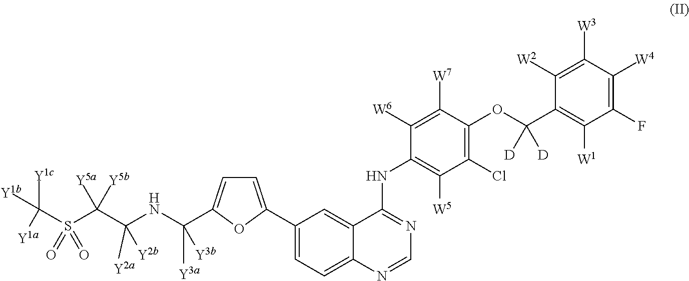
or salt thereof, wherein:
each W and each Y is independently selected from hydrogen and deuterium; and
at least one W is deuterium and at least one of Y and W is hydrogen.

7. The compound of claim 6, wherein each of W¹ and W² is deuterium.

8. The compound of claim 7, wherein each of W¹, W², W³ and W⁴ is deuterium.

9. The compound of claim 7 or 8, wherein each of W⁵, W⁶ and W⁷ is hydrogen.

10. The compound of claim 7 or 8, wherein each of W⁵, W⁶, and W⁷ is deuterium.

11. The compound of claim 6, wherein the compound is one of the following compounds:

| | | | | | | | | each of | | | |
|---|---|---|---|---|---|---|---|---|---|---|---|
| | | | | | | | | W¹, W², | each of | | |
| | | | | | | | | W³ and | W⁵, W⁶, | | |
| Cmpd | Y^{1a} | Y^{1b} | Y^{1c} | Y^{2a} | Y^{2b} | Y^{3a} | Y^{3b} | W⁴ | and W⁷ | Y^{5a} | Y^{5b} |
| 200 | H | H | H | H | H | H | H | D | H | H | H |
| 201 | H | H | H | D | D | D | D | D | H | H | H |
| 202 | D | D | D | D | D | D | D | D | H | D | D |
| 203 | D | D | D | D | D | H | H | D | H | D | D |
| 204 | H | H | H | H | H | D | D | D | H | H | H |
| 205 | H | H | H | D | D | H | H | D | H | H | H |
| 300 | H | H | H | H | H | H | H | H | D | H | H |
| 301 | H | H | H | D | D | D | D | H | D | H | H |
| 302 | D | D | D | D | D | D | D | H | D | D | D |
| 303 | D | D | D | D | D | H | H | H | D | D | D |
| 304 | H | H | H | H | H | D | D | H | D | H | H |
| 305 | H | H | H | D | D | H | H | H | D | H | H |
| 400 | H | H | H | H | H | H | H | D | D | H | H |
| 401 | H | H | H | D | D | D | D | D | D | H | H |
| 402 | D | D | D | D | D | H | H | D | D | D | D |
| 403 | H | H | H | H | H | D | D | D | D | H | H |
| 404 | H | H | H | D | D | H | H | D | D | H | H |

or a salt thereof.

12. The compound of claim 6 wherein the compound is a compound of Formula IIb: 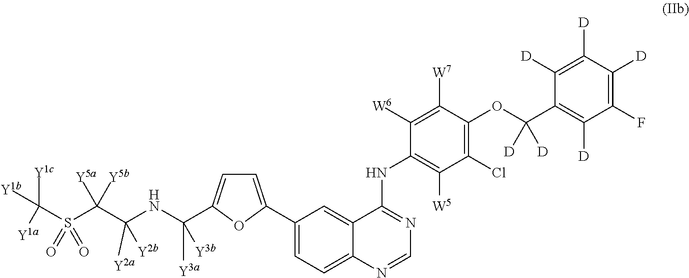
or a salt thereof, wherein
each W and each Y is independently selected from hydrogen and deuterium.

13. The compound of claim 12 wherein the compound is 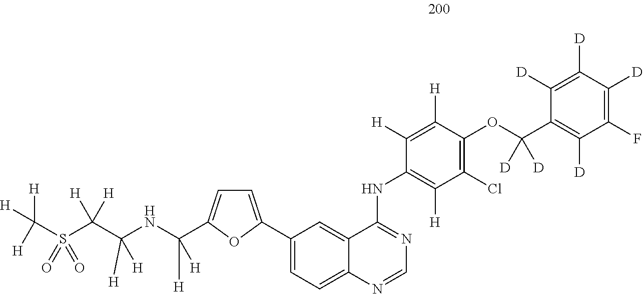
or a salt thereof.

14. The compound of claim 12 wherein the compound is a compound of Formula IIc: 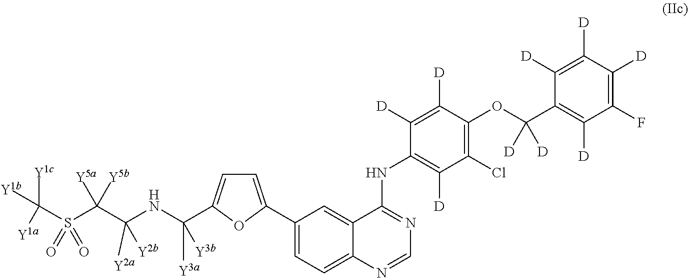
or a salt thereof wherein each Y is independently hydrogen or deuterium.

15. The compound of any one of claims 1 to 14, wherein any atom not designated as deuterium is present at its natural isotopic abundance.

16. A pharmaceutical composition comprising a compound of any one of the preceding claims or a pharmaceutically acceptable salt thereof; and a pharmaceutically acceptable carrier.

17. A method of inhibiting the tyrosine kinase activity of erbB-1 or erbB-2 in a cell comprising the step of contacting the cell with a compound of claim 1.

18. A method of treating a subject suffering from or susceptible to a neoplasia comprising the step of administering to the subject in need thereof an effective amount of a compound of pharmaceutically acceptable salt of the compound of claim 1 or of claim 6.

19. The method of claim 18, wherein the neoplasia is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, or a solid tumor such as a sarcoma or a carcinoma (fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, orretinoblastoma).

20. The method of claim 19, wherein the neoplasia is breast cancer, esophageal adenocarcinoma, esophageal squamous cell carcinoma, cervical cancer, head and neck cancer, solid tumors, non-Hodgkins' Lymphoma, gastric cancer, ovarian cancer, peritoneal cancer, brain and CNS tumors (glioma, glioblastoma multiforme, gliosarcoma), prostate cancer, endometrial cancer, colorectal cancer, non-small cell lung cancer, liver cancer, renal cancer, or pancreatic cancer.

21. The method of claim 19 or 20, wherein the neoplasia is erbB2-, erbB4-, or EGF-receptor positive.

22. The method of claim 21, wherein the neoplasia is erbB2-, or EGF-receptor positive.

23. The method of claim 22, wherein the neoplasia is breast cancer.

24. The compound or salt of the compound of claim 1, wherein the compound or salt of the compound or salt of the compound has an isotopic enrichment factor for each position designated as having a deuterium atom of at least 6000.

25. The compound or salt of the compound of claim 6, wherein the compound or salt of the compound has an isotopic enrichment factor for each position designated as having a deuterium atom of at least 6000.

26. The pharmaceutical composition of claim 16, wherein the compound or pharmaceutically acceptable salt of the compound has an isotopic enrichment factor for each position designated as having a deuterium atom of at least 6000.

27. The method of claim 18, wherein the compound or pharmaceutically acceptable salt of the compound has an isotopic enrichment factor for each position designated as having a deuterium atom of at least 6000.
